(19) United States
(12) Patent Application Publication
(10) Pub. No.: US 2025/0282743 A1
(43) Pub. Date: Sep. 11, 2025

(54) METHODS OF SYNTHESIZING CANNABIDIOL, DERIVATIVES THEREOF, AND OTHER PHYTOCANNABINOIDS

(71) Applicant: Citrachem Corp., Concord, MA (US)

(72) Inventor: Jason S. Kingsbury, Agoura Hills, CA (US)

(21) Appl. No.: 18/833,755

(22) PCT Filed: Jan. 27, 2023
(86) PCT No.: PCT/US2023/011743

§ 371 Date: Jul. 26, 2024

Related U.S. Application Data
(60) Provisional application No. 63/303,614, filed on Jan. 27, 2022.

(51) Int. Cl. C07D 311/80; A61K 31/00; C07C 37/16; C07C 37/50; C07C 45/29; C07C 67/343

(52) U.S. Cl. CPC C07D 311/80; A61K 31/658; C07C 37/16; C07C 37/50; C07C 45/292; C07C 67/343

(57) ABSTRACT

Provided herein are synthetic methods for the preparation of cannabidiol (CBD) and delta-9-tetrahydrocannabinol (THC) as well as derivatives thereof as racemic mixtures or in enantiopure form. Also provided herein are synthetic methods for the preparation of relevant intermediates in the process.

### RELATED APPLICATION

This application claims the benefit of and priority to U.S. Provisional Patent Application Ser. No. 63/303,614, filed Jan. 27, 2022, which is hereby incorporated by reference in its entirety.

### BACKGROUND

Over 100 compounds, commonly known as cannabinoids (Hanus, L. O. et al. 2016), have been found in the Cannabis sativa plant. Included among these natural products are cannabidiol (CBD) and delta-9-tetrahydrocannabinol (Δ⁹-THC). In the United States, the cannabidiol drug Epidiolex® was approved by the Food and Drug Administration in 2018 for the treatment of two rare epilepsy disorders, and the compound has been extensively studied for other conditions. Dronabinol, the generic name for delta-9-tetrahydrocannabinol, has widespread medical utility as an antiemetic agent (appetite stimulant) and a sleep apnea reliever. Clinical usage is also FDA-approved for the treatment of HIV/AIDS-induced anorexia, chemotherapy-induced nausea, and glaucoma. Many other cannabinoids are being investigated for various indications, including cannabidivarin for treatment of epilepsy and tetrahydrocannabidivarin for treatment of diabetes.

Many different routes to produce natural cannabinoids and related compounds have been reported (Mechoulam, R., McCallum, N. K., and Burstein, S. 1976; Aguillón, A. R. et al. 2021). Manufacture of CBD on commercial scale presents a number of challenges, particularly with regard to direct and convergent syntheses that rely on Friedel-Crafts alkylation of olivetol (1,3-dihydroxy-5-pentylbenzene) to forge the central aryl-monoterpenyl C—C bond. Since the reaction requires a strong Lewis or Bronsted acidic reagent to promote terpenylation, it can be impossible to prevent further cyclization of the phenolic hydroxyl group into the dihydropyran ring of Δ⁹-THC. Additional isomers such as cis-Δ⁹-THC (a dihydropyran stereoisomer), Δ⁸-THC (deriving from migration of the cyclohexene double bond), and iso-THC (a regioisomer resulting from F—C alkylation ortho to the n-pentyl group) form that are tedious to separate from the desired product. This adds time and costs to a number of reported syntheses (Petrzilka, T. et al. 1967; Baek, S-H., Srebnik, M., and Mechoulam, R. 1985), making it difficult to achieve current standards of purity for active pharmaceutical ingredients. Extraction and purification of naturally occurring CBD is economically less attractive and presents agricultural limitations. For example, even industrial strains of hemp that are bred specifically to contain low quantities of the psychoactive Δ⁹-THC can be found to exceed the legal limit of ≤0.3% by dry mass, owing to variability in growing or harvesting conditions. Alternative synthetic pathways are known that can limit contamination by THC's, but they rely on protected olivetol intermediates, and this makes the routes more lengthy (Aguillón, A. R. et al. 2021).

Given the rapid facility by which delta-9-tetrahydrocannabinol (Δ⁹-THC) can undergo acid-catalyzed isomerization, the most efficient chemical syntheses that have been disclosed tend to be based upon selective crystallization and isolation of a single Friedel-Crafts product, often in yields no higher than 35-45%. Souza et al. (2008) have reported a Δ⁹-THC synthesis featuring a late-stage dihydropyran annulation with mild zinc (II) chloride-promoted conditions (49% yield). What makes this strategy possible is the use of cis-menth-1-ene-3,8-diol as the terpene fragment, effectively masking the isopropenyl substituent found in CBD as a tertiary alcohol. By carrying the dihydropyran ring in latent form through the synthesis, the authors achieved a 39% yield in Friedel-Crafts alkylation of olivetol with camphorsulfonic acid (CSA) as promoter. There are, however, disadvantages to the route overall. The 10C cis-menth-1-ene-3,8-diol takes six linear steps to prepare from a propargylic alcohol and requires a resolution in order to obtain optically active material. In another route, Burdick et al. (2012) were also able to develop distinct reagents for Friedel-Crafts terpenylation and dihydropyran cyclization. Use of scandium (III) triflate for condensation of olivetol (and olivetolate esters) with a more commonly used terpene component, (±)-menthadienol, leads to crude ethyl cannibidiolate that is both hydrolyzed and decarboxylated at reflux to furnish 44% of solid CBD. The second innovation features tri-isobutylaluminum as a reagent for selective cyclization of CBD to a mixture of 95.6% Δ⁹-THC and 1.1% cis-Δ⁹-THC. But as in the aforementioned report, access to the monoterpene (±)-menthadienol requires multi-step synthesis, typically three to five operations from (±)-limonene oxide or (R)-limonene itself.

### SUMMARY

In certain embodiments, the present disclosure provides a process for producing a compound having the structure of formula (±)-(I):

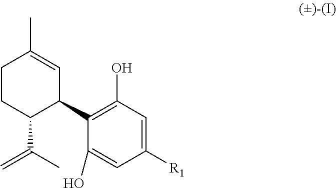

wherein
R₁ is alkyl,
the process comprising:
(i) reacting citral with a Lewis acid in a suitable solvent under conditions sufficient to produce a compound having the structure of formula (±)-(IIa):

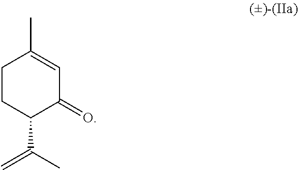

In certain embodiments, the present disclosure also provides a process for producing a compound having the structure of formula (±)-(Ia):

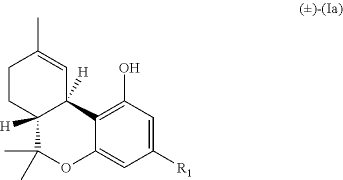

wherein
R₁ is alkyl,
the process comprising:
(i) reacting citral with a Lewis acid in a suitable solvent under conditions sufficient to produce a compound having the structure of formula (±)-(IIa):

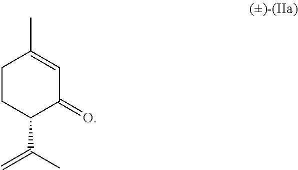

In certain embodiments, the present disclosure further provides a process for producing a compound having the structure of formula (±)-(IIa):

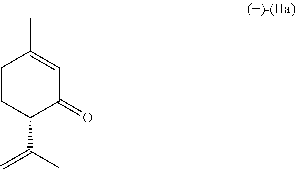

the process comprising reacting citral with a Lewis acid in a suitable solvent under conditions sufficient to produce the compound.

In certain embodiments, the present disclosure further provides a process for producing a compound having the structure of formula (−)-(I):

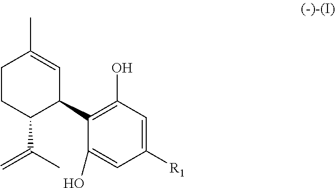

wherein
R₁ is alkyl,
the process comprising:
(i) reacting (R)-limonene with an oxidizing agent in a first suitable solvent under conditions sufficient to produce a compound having the structure of formula (±)-(IIa):

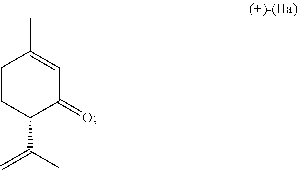

and
(ii) reacting the compound having the structure of formula (IIa) produced in step (i) with a reducing agent in a second suitable solvent under conditions sufficient to produce a compound having the structure of formula (+)-(II′) as a single enantiomer or (+)-(II″) as a single enantiomer:

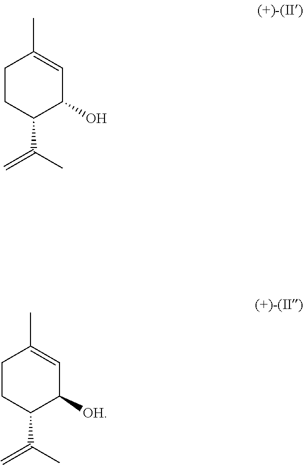

In certain embodiments, the present disclosure further provides a process for producing a compound having the structure of formula (−)-(Ia):

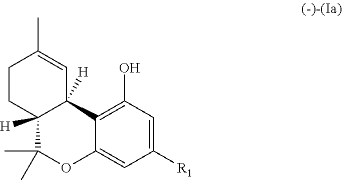

wherein
R₁ is alkyl,
the process comprising:
(i) reacting (R)-limonene with an oxidizing agent in a first suitable solvent under conditions sufficient to produce a compound having the structure of formula (+)-(IIa):

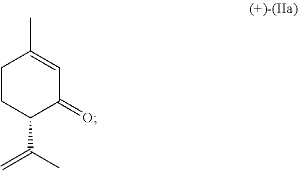

and
(ii) reacting the compound having the structure of formula (+)-(IIa) produced in step (i) with a reducing agent in a second suitable solvent under conditions sufficient to produce a compound having the structure of formula (+)-(II′) as a single enantiomer or (+)-(II″) as a single enantiomer:

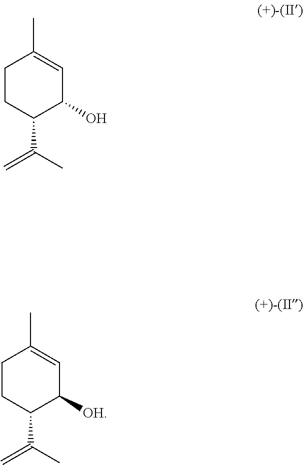

In certain embodiments, the present disclosure further provides a process for producing a compound having the structure of formula (+)-(II′) or (+)-(II″):

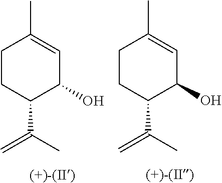

the process comprising:
(i) reacting (R)-limonene with an oxidizing agent in a first suitable solvent under conditions sufficient to produce a compound having the structure of formula (+)-(IIa):

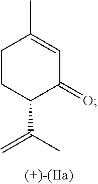

and
(ii) reacting the compound having the structure of formula (+)-(IIa) produced in step (i) with a reducing agent in a second suitable solvent under conditions sufficient to produce a compound having the structure of formula (+)-(II′) as a single enantiomer or (+)-(II″) as a single enantiomer.

In certain embodiments, the present disclosure further provides a process for producing a compound having the structure of formula (III):

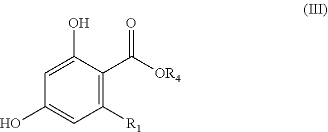

wherein
R₁ is alkyl; and
R₄ is alkyl,
the process comprising:
(i) reacting the compound having the structure of formula (VII):

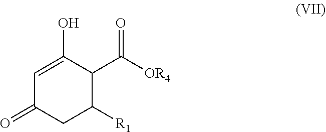

with Br₂ in a first suitable solvent under conditions sufficient to produce the compound having the structure of formula (VIIa):

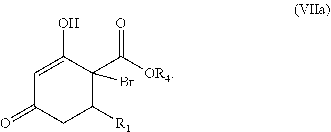

In certain embodiments, the present disclosure further provides a process for producing a compound having the structure of formula (III):

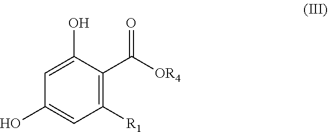

wherein
R₁ is alkyl; and
R₄ is alkyl,
the process comprising:
(b) reacting the compound having the structure of formula (VIb)

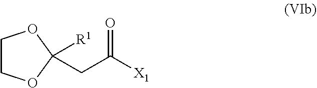

wherein
X₁ is a halide or activating ester moiety,
with a diene having the structure of formula (VIc) in the presence of a Lewis acid in a suitable solvent:

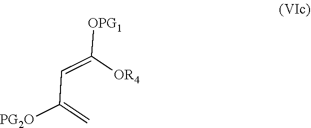

wherein
PG₁ and PG₂ are each independently a hydroxyl protecting group; and
R₄ is alkyl, under conditions sufficient to form the cyclized compound having the structure of formula (III).

In certain embodiments, the present disclosure further provides a process for producing a compound having the structure of formula (VIII):

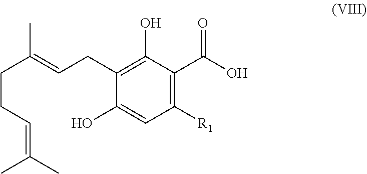

wherein
R₁ is alkyl; and
R₄ is alkyl,
the process comprising:
(i) reacting the compound having the structure of formula (III):

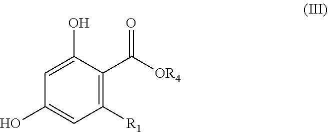

with geraniol in the presence of an acid in a first suitable solvent under conditions sufficient to produce the compound having the structure of formula (VIIIa):

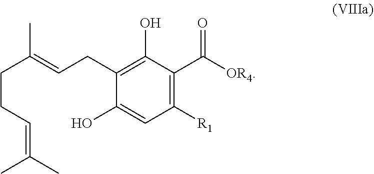

### BRIEF DESCRIPTION OF THE DRAWINGS

FIG. 1: General synthetic routes to cannabidiol (CBD).

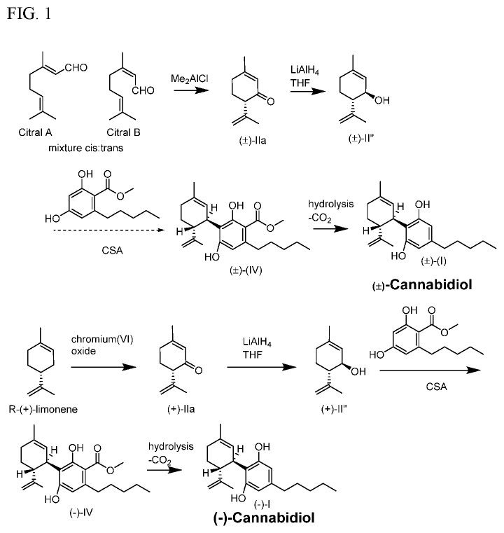

FIG. 2: General synthetic routes to delta-9-tetrahydrocannabinol (Δ⁹-THC).

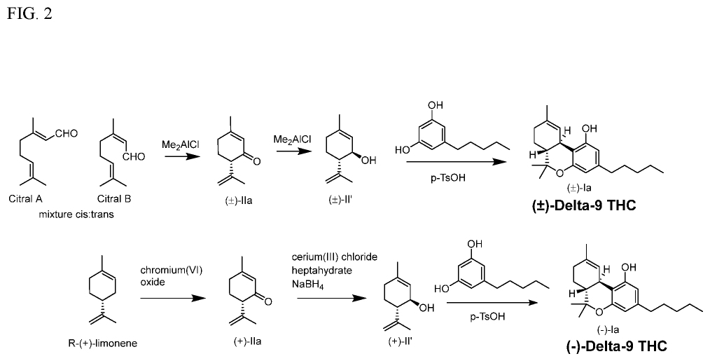

FIG. 3: Example synthetic route to cannabigerolic acid (CBGA).

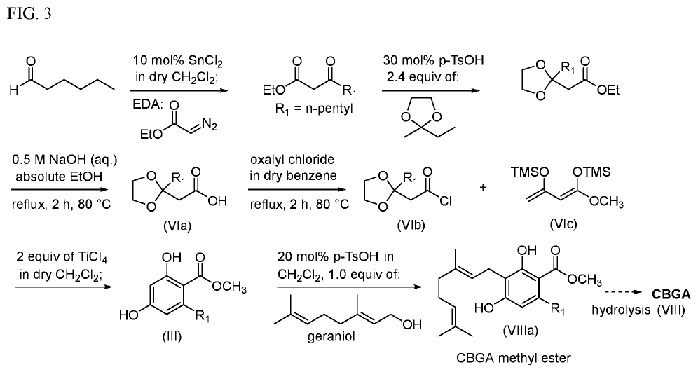

### DETAILED DESCRIPTION

The present invention is directed towards overcoming inefficiencies in the preparation of phytocannabinoids. Specifically, methods are disclosed for preparing CBD in a stereo- and regiochemically controlled manner from cannabidioloic acid (CBDA) as the penultimate precursor, thus mirroring how the metabolite is biosynthesized in Nature. In some embodiments, the present pathway of synthesis provides a stereoenriched monoterpenoid for the Friedel-Crafts alkylation in just one-two steps from an achiral and widely abundant starting material. Disclosed herein are improved synthetic routes to, for example, Cannabidiol (CBD) and delta-9-Tetrahydrocannabinol (Δ⁹-THC) in racemic or enantiopure form. Starting from readily available and inexpensive starting materials, e.g. citral and limonene, the routes are scalable and cost effective. Also disclosed are synthetic routes to key intermediates, e.g., trans-isopiperitenol and methyl olivetolate.

### Processes

In certain embodiments, the invention relates to a process for producing a compound having the structure of formula (±)-(I):

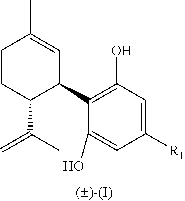

wherein
R₁ is alkyl,
the process comprising:
(i) reacting citral with a Lewis acid in a suitable solvent under conditions sufficient to produce a compound having the structure of formula (±)-(II′):

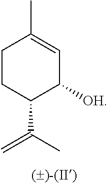

In certain embodiments, the invention relates to a process for producing a compound having the structure of formula (±)-(I):

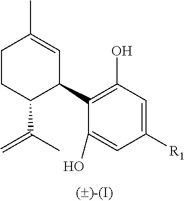

wherein
R₁ is alkyl,
the process comprising:
(i) reacting citral with a Lewis acid in a suitable solvent under conditions sufficient to produce a compound having the structure of formula (±)-(II″):

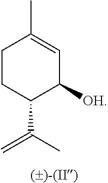

In certain embodiments, the invention relates to a process for producing a compound having the structure of formula (±)-(I):

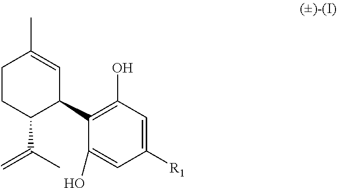

wherein
R₁ is alkyl,
the process comprising:
(i) reacting citral with a Lewis acid in a suitable solvent under conditions sufficient to produce a compound having the structure of formula (±)-(IIa):

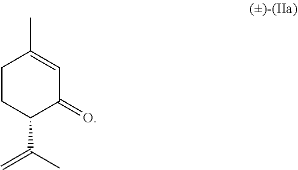

In certain embodiments, the citral is a mixture of citral A and citral B. In other embodiments, the citral is citral B.

In certain embodiments, the Lewis acid comprises an aluminum metal center.

In certain embodiments, the Lewis acid comprises an aluminum (III) salt.

In certain embodiments, the Lewis acid is a dialkyl aluminum chloride.

In certain embodiments, the Lewis acid is dimethyl aluminum chloride. In other embodiments, the Lewis acid is diethyl aluminum chloride.

In certain embodiments, a solution of the citral in the suitable solvent and a solution of the Lewis acid in the suitable solvent are slowly mixed together over 2 to 4 hours.

In certain embodiments, the mixing occurs at a temperature of less than 0° C.

In certain embodiments, the mixing occurs at a temperature of about −10° C. In other embodiments, the mixing occurs at a temperature of about −20 to 0° C.

In certain embodiments, the suitable solvent is dichloromethane. In some embodiments, the suitable solvent is chloroform.

In certain embodiments, the process further comprises:
(ia) reacting the compound having the structure of formula (±)-(IIa) produced in step (i) with a reducing agent in a second suitable solvent under conditions sufficient to produce a compound having the structure of formula (±)-(II″):

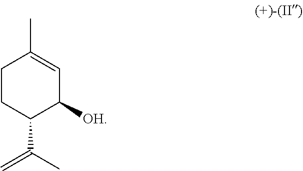

In certain embodiments, the reducing agent comprises lithium metal.

In certain embodiments, the reducing agent is lithium aluminum hydride.

In certain embodiments, the second suitable solvent is tetrahydrofuran.

In certain embodiments, the reaction of step (ii) occurs at a temperature of less than 0° C.

In certain embodiments, the process further comprises:
(ii) reacting the compound having the structure of formula (±)-(II) produced in step (i) with a compound having the structure of formula (III) in the presence of an acid in a second suitable solvent:

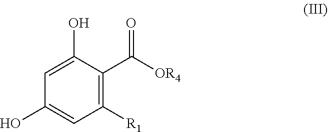

wherein R₄ is alkyl, under conditions sufficient to produce a compound having the structure of formula (±)-(IV):

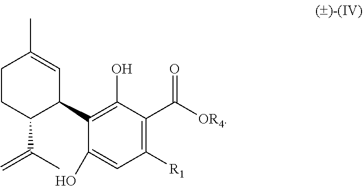

In certain embodiments, the acid is an organic acid or a Lewis acid. In certain embodiments, the acid is an organic acid. In certain embodiments, the organic acid is camphorsulfonic acid.

In certain embodiments, the second suitable solvent is dichloromethane. In some embodiments, the second suitable solvent is chloroform.

In certain embodiments, the process further comprises:
(iii) subjecting the compound having the structure of formula (±)-(IV) produced in step (ii) to basic hydrolysis conditions sufficient to produce a compound having the structure of formula (±)-(V):

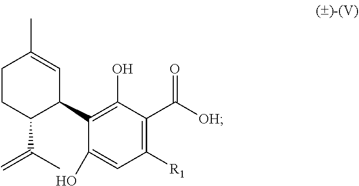

and
(iv) subjecting the compound having the structure of formula (±)-(V) produced in step (iii) to decarboxylation conditions sufficient to produce the compound having the structure of formula (±)-(I).

In certain embodiments, the compound having the structure of formula (III) is prepared by a process comprising:
(a) reacting a compound having the structure of formula (VIa):

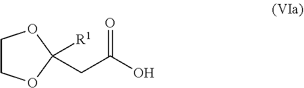

with an activating agent in a first suitable solvent under conditions sufficient to produce a compound having the structure of formula (VIb):

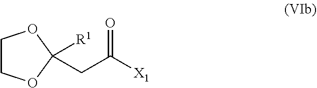

wherein
X₁ is a halide or activating ester moiety;
(b) reacting the compound having the structure of formula (VIb) with a diene having the structure of formula (VIc) in the presence of a Lewis acid in a second suitable solvent:

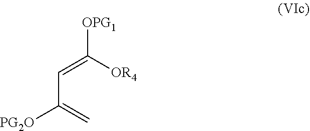

wherein
PG₁ and PG₂ are each independently a hydroxyl protecting group; and
R₄ is alkyl, under conditions sufficient to form the cyclized compound having the structure of formula (III).

In certain embodiments, X₁ is a Cl.

In certain embodiments, R₁ is unsubstituted alkyl. In other embodiments, R₁ is substituted alkyl.

In certain embodiments, R₁ is C₄-C₆ alkyl. In other embodiments, R₁ is C₇-C₁₀ alkyl.

In certain embodiments, R₁ is n-pentyl.

In certain embodiments, the invention also relates to a process for producing a compound having the structure of formula (±)-(Ia):

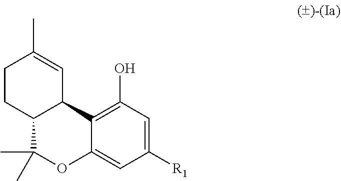

wherein
R₁ is alkyl,
the process comprising:
(i) reacting citral with a Lewis acid in a suitable solvent under conditions sufficient to produce a compound having the structure of formula (±)-(II′):

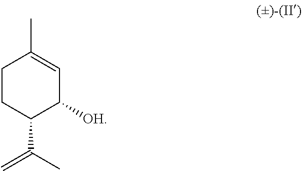

In certain embodiments, the invention relates to a process for producing a compound having the structure of formula (±)-(Ia):

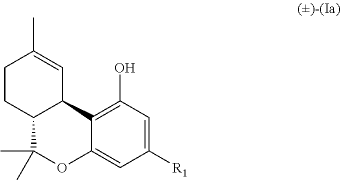

wherein
R₁ is alkyl,
the process comprising:
(i) reacting citral with a Lewis acid in a suitable solvent under conditions sufficient to produce a compound having the structure of formula (±)-(II″):

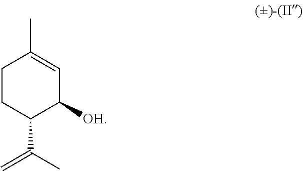

In certain embodiments, the invention relates to a compound having the structure of formula (±)-(Ia):

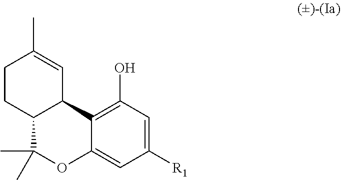

wherein
R₁ is alkyl,
the process comprising:
(i) reacting citral with a Lewis acid in a suitable solvent under conditions sufficient to produce a compound having the structure of formula (±)-(IIa):

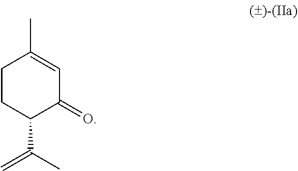

In certain embodiments, the citral is a mixture of citral A and citral B.

In certain embodiments, the citral is citral B.

In certain embodiments, the Lewis acid comprises an aluminum metal center.

In certain embodiments, the Lewis acid comprises an aluminum (III) salt.

In certain embodiments, the Lewis acid is a dialkyl aluminum chloride.

In certain embodiments, the Lewis acid is dimethyl aluminum chloride. In some embodiments, the Lewis acid is diethyl aluminum chloride.

In certain embodiments, a solution of citral in the suitable solvent and a solution of the Lewis acid in the suitable solvent are slowly mixed together over 2 to 4 hours.

In certain embodiments, the mixing occurs at a temperature of less than 0° C.

In certain embodiments, the mixing occurs at a temperature of about −10° C. In some embodiments, the mixing occurs at a temperature of about −20 to 0° C.

In certain embodiments, the suitable solvent is dichloromethane. In some embodiments, the suitable solvent is chloroform.

In certain embodiments, the process further comprises:
(ia) reacting the compound having the structure of formula (±)-(IIa) produced in step (i) with a reducing agent in a second suitable solvent under conditions sufficient to produce a compound having the structure of formula (+)-(II″):

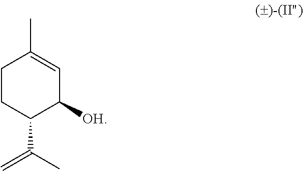

In certain embodiments, the reducing agent comprises lithium metal.

In certain embodiments, the reducing agent is lithium aluminum hydride.

In certain embodiments, the second suitable solvent is tetrahydrofuran.

In certain embodiments, the reaction of step (ii) occurs at a temperature of less than 0° C. In certain embodiments, the process further comprises:
(ii) reacting the compound having the structure of formula (±)-(II) produced in step (i) with a compound having the structure of formula (IIIa) in the presence of an acid in a second suitable solvent:

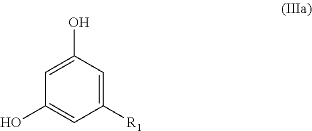

thereby producing the compound having the structure of formula (±)-(Ia).

In certain embodiments, the acid is an organic acid or a Lewis acid. In certain embodiments, the acid is an organic acid. In certain embodiments, the acid is p-toluenesulfonic acid.

In certain embodiments, the suitable solvent is dichloromethane.

In certain embodiments, R₁ is unsubstituted alkyl. In other embodiments, R₁ is substituted alkyl.

In certain embodiments, R₁ is C₄-C₆ alkyl. In other embodiments, R₁ is C₇-C₁₀ alkyl.

In certain embodiments, R₁ is n-pentyl.

In certain embodiments, the invention further relates to a process for producing a compound having the structure of formula (±)-(IIa):

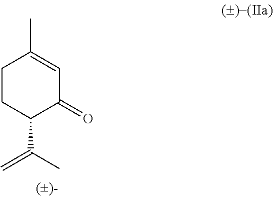

the process comprising reacting citral with a Lewis acid in a suitable solvent under conditions sufficient to produce the compound.

In certain embodiments, the citral is a mixture of citral A and citral B.

In certain embodiments, the citral is citral B.

In certain embodiments, the Lewis acid comprises an aluminum metal center.

In certain embodiments, the Lewis acid comprises an aluminum (III) salt.

In certain embodiments, the Lewis acid is a dialkyl aluminum chloride.

In certain embodiments, the Lewis acid is dimethyl aluminum chloride. In some embodiments, the Lewis acid is diethyl aluminum chloride.

In certain embodiments, a solution of citral in the suitable solvent and a solution of the Lewis acid in the suitable solvent are slowly mixed together over 2 to 4 hours.

In certain embodiments, the mixing occurs at a temperature of less than 0° C.

In certain embodiments, the mixing occurs at a temperature of about −10° C. In some embodiments, the mixing occurs at a temperature of about −20 to 0° C.

In certain embodiments, the suitable solvent is dichloromethane. In some embodiments, the suitable solvent is chloroform.

In certain embodiments, the invention relates to a process for producing a compound having the structure of formula (±)-(II″):

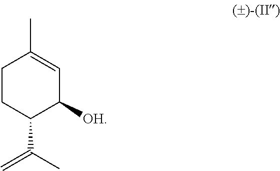

the process comprising preparing the compound having the structure of formula (±)-(IIa) according to the process disclosed herein; and reacting the compound having the structure of formula (±)-(IIa) with a reducing agent in a second suitable solvent under conditions sufficient to produce the compound having the structure of formula (+)-(II″).

In certain embodiments, the reducing agent comprises lithium metal.

In certain embodiments, the reducing agent is lithium aluminum hydride.

In certain embodiments, the second suitable solvent is tetrahydrofuran.

In certain embodiments, the reaction of step (ii) occurs at a temperature of less than 0° C.

In certain embodiments, the invention further provides a method for producing a compound having the structure of formula (−)-(I):

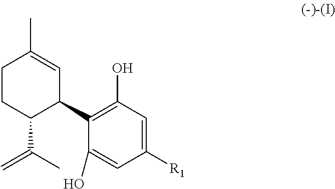

wherein
R₁ is alkyl,
the process comprising:
(i) reacting (R)-limonene with an oxidizing agent in a first suitable solvent under conditions sufficient to produce a compound having the structure of formula (+)-(IIa):

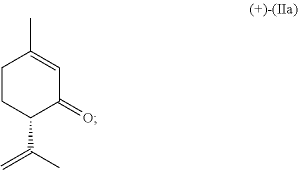

and
(ii) reacting the compound having the structure of formula (+)-(IIa) produced in step (i) with a reducing agent in a second suitable solvent under conditions sufficient to produce a compound having the structure of formula (+)-(II′) as a single enantiomer:

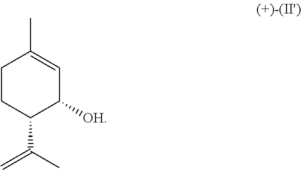

In certain embodiments, the invention provides a method for producing a compound having the structure of formula (−)-(I):

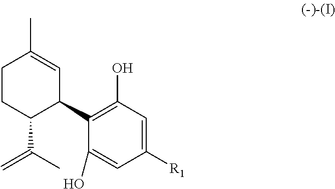

wherein
R₁ is alkyl,
the process comprising:
(i) reacting (R)-limonene with an oxidizing agent in a first suitable solvent under conditions sufficient to produce a compound having the structure of formula (+)-(IIa):

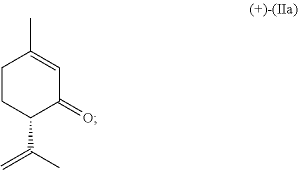

and
(ii) reacting the compound having the structure of formula (+)-(IIa) produced in step (i) with a reducing agent in a second suitable solvent under conditions sufficient to produce a compound having the structure of formula (+)-(II″) as a single enantiomer:

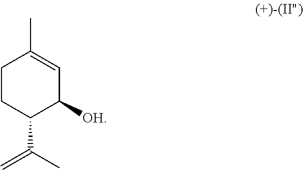

In certain embodiments, the oxidizing agent comprises a chromium metal complex or salt.

In certain embodiments, the oxidizing agent is chromium (VI) oxide.

In certain embodiments, the first suitable solvent is chloroform.

In certain embodiments, the reducing agent comprises sodium metal.

In certain embodiments, the reducing agent is sodium borohydride.

In certain embodiments, the second suitable solvent is methanol.

In certain embodiments, the reaction of step (ii) occurs in the presence of cerium (III) chloride or samarium (III) iodide.

In certain embodiments, the reducing agent comprises lithium metal.

In certain embodiments, the reducing agent is lithium aluminum hydride.

In certain embodiments, the second suitable solvent is tetrahydrofuran.

In certain embodiments, the reaction of step (ii) occurs at a temperature of less than 0° C.

In certain embodiments, the process further comprises:

(iii) reacting the compound having the structure of formula (+)-(II′) or (+)-(II″) produced in step (ii) with a compound having the structure of formula (III) in the presence of an acid in a suitable solvent:

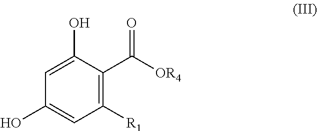

wherein R₄ is alkyl, under conditions sufficient to produce a compound having the structure of formula (−)-(IV):

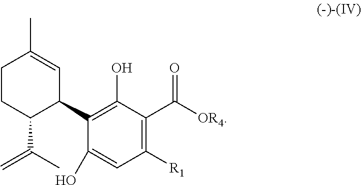

In certain embodiments, the acid is an organic acid or Lewis acid. In certain embodiments, the acid is an organic acid. In certain embodiments, the acid is camphorsulfonic acid.

In certain embodiments the suitable solvent is dichloromethane.

In certain embodiments, the process further comprises:
(iv) subjecting the compound having the structure of formula (−)-(IV) produced in step (iii) to basic hydrolysis conditions sufficient to produce a compound having the structure of formula (−)-(V):

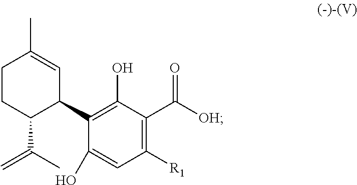

and
(v) subjecting the compound having the structure of formula (−)-(V) produced in step (iv) to decarboxylation conditions sufficient to produce the compound having the structure of formula (−)-(I).

In certain embodiments, the compound having the structure of formula (III) is prepared by a process comprising:
(a) reacting a compound having the structure of formula (VIa):

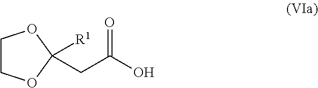

with an activating agent in a first suitable solvent under conditions sufficient to produce an activated ester compound having the structure of formula (VIb):

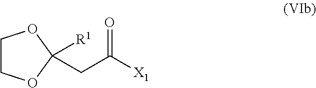

wherein
X₁ is a halide or activating ester moiety;
(b) reacting the compound having the structure of formula (VIb) with a diene having the structure of formula (II) in the presence of a Lewis acid in a second suitable solvent:

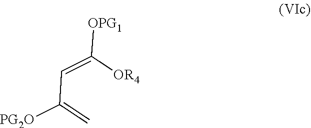

wherein
PG₁ and PG₂ are each independently a hydroxyl protecting group; and
R₄ is alkyl, under conditions sufficient to form the cyclized compound having the structure of formula (III).

In certain embodiments, X₁ is Cl.

In certain embodiments, R₁ is unsubstituted alkyl. In other embodiments, Riis substituted alkyl.

In certain embodiments, R₁ is C₄-C₆ alkyl. In other embodiments, R₁ is C₇-C₁₀ alkyl.

In certain embodiments, R₁ is n-pentyl.

In certain embodiments, the invention further provides a process for producing a compound having the structure of formula (−)-(Ia):

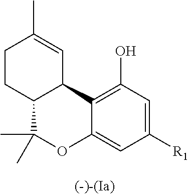

wherein
R₁ is alkyl,
the process comprising:
(i) reacting (R)-limonene with an oxidizing agent in a first suitable solvent under conditions sufficient to produce a compound having the structure of formula (+)-(IIa):

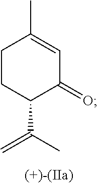

and
(ii) reacting the compound having the structure of formula (+)-(IIa) produced in step (i) with a reducing agent in a second suitable solvent under conditions sufficient to produce a compound having the structure of formula (+)-(II′) as a single enantiomer:

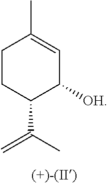

In certain embodiments, the invention provides a process for producing a compound having the structure of formula (−)-(Ia):

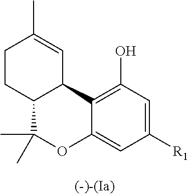

wherein
R₁ is alkyl,
the process comprising:
(i) reacting (R)-limonene with an oxidizing agent in a first suitable solvent under conditions sufficient to produce a compound having the structure of formula (+)-(IIa):

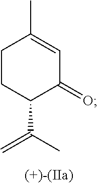

and
(ii) reacting the compound having the structure of formula (+)-(IIa) produced in step (i) with a reducing agent in a second suitable solvent under conditions sufficient to produce a compound having the structure of formula (+)-(II″) as a single enantiomer:

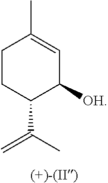

In certain embodiments, the oxidizing agent comprises a chromium metal complex or salt.

In certain embodiments, the oxidizing agent is chromium (VI) oxide.

In certain embodiments, the first suitable solvent is chloroform.

In certain embodiments, the reducing agent comprises sodium metal.

In certain embodiments, the reducing agent is sodium borohydride.

In certain embodiments, the second suitable solvent is methanol.

In certain embodiments, the reaction of step (ii) occurs in the presence of cerium (III) chloride or samarium (III) iodide.

In certain embodiments, the reducing agent comprises lithium metal.

In certain embodiments, the reducing agent is lithium aluminum hydride.

In certain embodiments, the second suitable solvent is tetrahydrofuran.

In certain embodiments, the reaction of step (ii) occurs at a temperature of less than 0° C.

In certain embodiments, the process further comprises:
(iii) reacting the compound having the structure of formula (+)-(II) produced in step (ii) with a compound having the structure of formula (IIIa) in the presence of an organic acid or a Lewis acid in in a second suitable solvent:

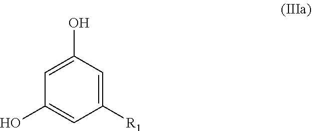

so as to thereby produce the compound having the structure of formula (−)-(Ia).

In certain embodiments, the acid is an organic acid or a Lewis acid.

In certain embodiments, the acid is p-toluenesulfonic acid.

In certain embodiments, the suitable solvent is dichloromethane.

In certain embodiments, R₁ is unsubstituted alkyl. In other embodiments, Riis substituted alkyl.

In certain embodiments, R₁ is C₄-C₆ alkyl. In other embodiments, R₁ is C₇-C₁₀ alkyl.

In certain embodiments, R₁ is n-pentyl.

In certain embodiments, the invention further provides a process for producing a compound having the structure of formula (±)-(II′):

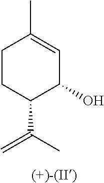

the process comprising:
(i) reacting (R)-limonene with an oxidizing agent in a first suitable solvent under conditions sufficient to produce a compound having the structure of formula (IIa):

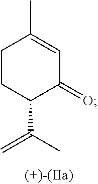

and
(ii) reacting the compound having the structure of formula (+)-(IIa) produced in step (i) with a reducing agent in a second suitable solvent under conditions sufficient to produce a compound having the structure of formula (+)-(II′) as a single enantiomer.

In certain embodiments, the invention provides a process for producing a compound having the structure of formula (+)-(II″):

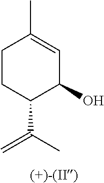

the process comprising:
(i) reacting (R)-limonene with an oxidizing agent in a first suitable solvent under conditions sufficient to produce a compound having the structure of formula (+)-(IIa):

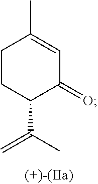

and
(ii) reacting the compound having the structure of formula (+)-(IIa) produced in step (i) with a reducing agent in a second suitable solvent under conditions sufficient to produce a compound having the structure of formula (+)-(II″) as a single enantiomer.

In certain embodiments, the oxidizing agent comprises a chromium metal complex or salt.

In certain embodiments, the oxidizing agent is chromium (VI) oxide.

In certain embodiments, the first suitable solvent is chloroform.

In certain embodiments, the reducing agent comprises sodium metal.

In certain embodiments, the reducing agent is sodium borohydride.

In certain embodiments, the second suitable solvent is methanol.

In certain embodiments, the reaction of step (ii) occurs in the presence of cerium (III) chloride or samarium (III) iodide.

In certain embodiments, the reducing agent comprises lithium metal.

In certain embodiments, the reducing agent is lithium aluminum hydride.

In certain embodiments, the second suitable solvent is tetrahydrofuran.

In certain embodiments, the reaction of step (ii) occurs at a temperature of less than 0° C.

In certain embodiments, the invention further provides a process for producing a compound having the structure of formula (III):

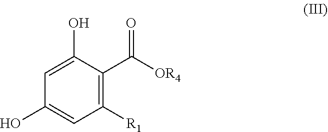

wherein
R₁ is alkyl; and
R₄ is alkyl,
the process comprising:
(i) reacting the compound having the structure of formula (VII):

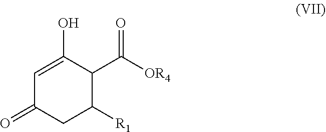

with Br₂ in a first suitable solvent under conditions sufficient to produce the compound having the structure of formula (VIIa):

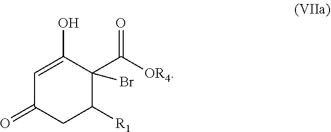

In certain embodiments, the first suitable solvent is dimethylformamide.

In certain embodiments, the reaction occurs at a temperature of −10 to 10° C.

In certain embodiments, about 1 molar equivalent of Br₂ is employed in the reaction.

In certain embodiments, about 1 to 1.05 molar equivalent of Br₂ is employed in the reaction.

In certain embodiments, the process further comprises:
(ii) refluxing the compound having the structure of formula (VIIa) produced in step (i) in a second suitable solvent under conditions sufficient to produce a compound having the structure of formula (III).

In certain embodiments, the second suitable solvent is toluene.

In certain embodiments, the compound having the structure of formula (VII) is prepared by a process comprising reacting a dialkyl malonate with trans-3-nonen-2-one in the presence of a base in a third suitable solvent under conditions sufficient to produce the compound having the structure of formula (VII).

In certain embodiments, the dialkyl malonate is dimethyl malonate.

In certain embodiments, the base is sodium methoxide. In some embodiments, the base is sodium ethoxide.

In certain embodiments, the third suitable solvent is methanol. In some embodiments, the third suitable solvent is ethanol.

In certain embodiments, the reaction occurs at reflux.

In certain embodiments, the invention provides a process for producing a compound having the structure of formula (III):

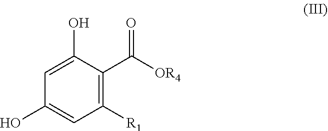

wherein
R₁ is alkyl; and
R₄ is alkyl,
the process comprising:
(b) reacting the compound having the structure of formula (VIb)

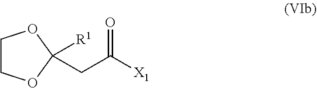

wherein
X₁ is a halide or activating ester moiety,
with a diene having the structure of formula (VIc) in the presence of a Lewis acid in a suitable solvent:

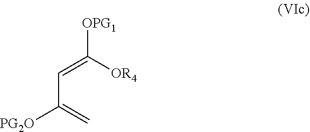

wherein
PG₁ and PG₂ are each independently a hydroxyl protecting group; and
R₄ is alkyl, under conditions sufficient to form the cyclized compound having the structure of formula (III).

In certain embodiments, the method further comprises:
(a) reacting a compound having the structure of formula (VIa):

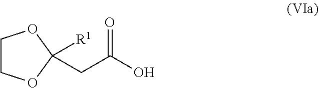

with an activating agent in a first suitable solvent under conditions sufficient to produce a compound having the structure of formula (VIb):

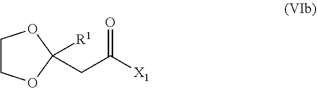

wherein
X₁ is a halide or activating ester moiety.

In certain embodiments, in step (a) the activating agent is oxalyl chloride.

In certain embodiments, the first suitable solvent is benzene.

In certain embodiments, in step (b) the Lewis acid is titanium chloride.

In certain embodiments, the suitable solvent is dichloromethane.

In certain embodiments, R₄ is methyl. In other embodiments, the R₄ is ethyl.

In certain embodiments, R₁ is unsubstituted alkyl. In other embodiments, R₁ is substituted alkyl.

In certain embodiments, R₁ is C₄-C₆ alkyl. In other embodiments, R₁ is C₇-C₁₀ alkyl.

In certain embodiments, R₁ is n-pentyl.

In certain embodiments, the invention further provides a process for producing a compound having the structure of formula (VIII):

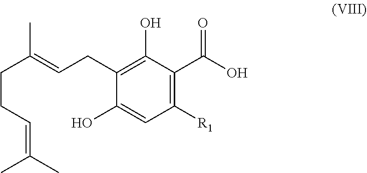

wherein
R₁ is alkyl; and
R₄ is alkyl,
the process comprising:
(i) reacting the compound having the structure of formula (III):

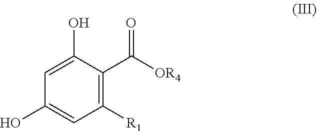

with geraniol in the presence of an acid in a first suitable solvent under conditions sufficient to produce the compound having the structure of formula (VIIIa):

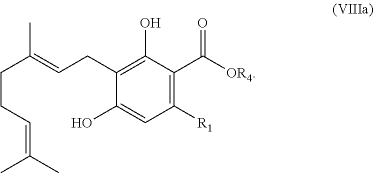

In certain embodiments, the compound of formula (III) in the above process is produced according to the process involving intermediates (VII) and (VIIa).

In certain embodiments, the compound of formula (III) in the above process is produced according to the process involving intermediates (VIb) and (VIc).

In certain embodiments, the acid is an organic acid or a Lewis acid.

In certain embodiments, the acid is camphorsulfonic acid.

In certain embodiments, the first suitable solvent is dichloromethane.

In certain embodiments, the reaction occurs at a temperature of 0 to 25° C.

In certain embodiments, the process further comprising:
(ii) subjecting the compound having the structure of formula (VIIIa) prepared in step (i) to decarboxylation conditions sufficient to produce the compound having the structure of formula (VIII).

In certain embodiments, R₄ is methyl. In certain embodiments, R₄ is ethyl.

In certain embodiments, R₁ is unsubstituted alkyl. In other embodiments, Riis substituted alkyl.

In certain embodiments, R₁ is C₄-C₆ alkyl. In other embodiments, R₁ is C₇-C₁₀ alkyl.

In certain embodiments, R₁ is n-pentyl.

In certain embodiments, the compound produced has the structure:

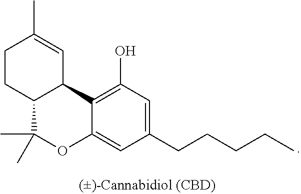 [SMILES: CCCCCc1cc(O)c2c(c1)OC(C)(C)[C@@H]1CCC(C)=C[C@@H]21]

In certain embodiments, the compound produced has the structure:

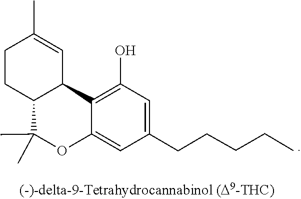 [SMILES: CCCCCc1cc(O)c2c(c1)OC(C)(C)[C@@H]1CCC(C)=C[C@@H]21]

In certain embodiments, the compound produced has the structure:

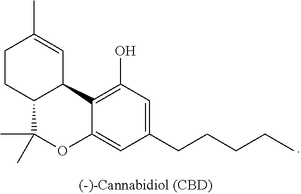 [SMILES: CCCCCc1cc(O)c2c(c1)OC(C)(C)[C@@H]1CCC(C)=C[C@@H]21]

In certain embodiments, the compound produced has the structure:

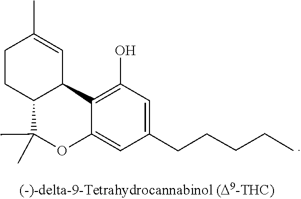 [SMILES: CCCCCc1cc(O)c2c(c1)OC(C)(C)[C@@H]1CCC(C)=C[C@@H]21]

In certain embodiments, the compound produced has the structure:

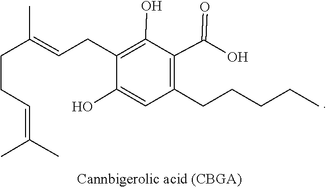 [SMILES: CCCCCc1cc(O)c(C/C=C(\C)CCC=C(C)C)c(O)c1C(=O)O]

In certain embodiments, the disclosure relates to a process for preparing (±)-cannabidiol (CBD), wherein the process comprises a preparation of (±)-isopiperitenone according to the present invention.

In certain embodiments, the disclosure relates to a process for preparing (±)-delta-9-tetrahydrocannabinol (Δ⁹-THC), wherein the process comprises a preparation of (±)-isopiperitenone according to the present invention.

In certain embodiments, the disclosure relates to a process for preparing (±)-cannabidiol (CBD), wherein the process comprises a preparation of (±)-isopipertenol according to the present invention.

In certain embodiments, the disclosure relates to a process for preparing (±)-delta-9-tetrahydrocannabinol (Δ⁹-THC), wherein the process comprises a preparation of (±)-isopipertenol according to the present invention.

In certain embodiments, the disclosure relates to a process for preparing (−)-cannabidiol (CBD), wherein the process comprises a preparation of (±)-trans-isopiperitenol according to the present invention.

In certain embodiments, the disclosure relates to a process for preparing (−)-delta-9-tetrahydrocannabinol (Δ⁹-THC), wherein the process comprises a preparation of (±)-trans-isopiperitenol according to the present invention.

In certain embodiments, the disclosure relates to a process for preparing (−)-cannabidiol (CBD), wherein the process comprises a preparation of (±)-cis-isopiperitenol according to the present invention.

In certain embodiments, the disclosure relates to a process for preparing (−)-delta-9-tetrahydrocannabinol (Δ⁹-THC), wherein the process comprises a preparation of (±)-cis-isopiperitenol according to the present invention.

In certain embodiments, the disclosure relates to a pharmaceutical composition comprising (±)-cannabidiol (CBD) and a pharmaceutically acceptable carrier, wherein the (±)-cannabidiol (CBD) is produced according to the present invention.

In certain embodiments, the disclosure relates to a pharmaceutical composition comprising (±)-delta-9-tetrahydrocannabinol (Δ⁹-THC) and a pharmaceutically acceptable carrier, wherein the (±)-delta-9-tetrahydrocannabinol (Δ⁹-THC) is produced according to the present invention.

In certain embodiments of any of the disclosed compounds, R₁ is C₄-C₆ alkyl. In other embodiments, R₁ is C₇-C₁₀ alkyl. In other embodiments of any of the disclosed compounds, R₁ is n-pentyl.

### Definitions

The terms “citral” as used herein means E-isomer Geranial (trans-Citral) or Citral A and/or the Z-isomer Neral (cis-Citral) or Citral B. Citral may refer to a mixture of the two isomers or each individual isomer. Generally, citral is commercially available as a mixture of the two isomers:

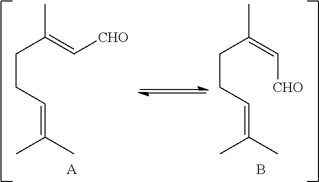

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N. Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

The starting materials and reagents used for the synthesis of the compounds described herein are synthesized or are obtained from commercial sources, such as, but not limited to, Sigma-Aldrich, Fisher Scientific (Fisher Chemicals), and Acros Organics.

All of the above, and any other publications, patents and published patent applications referred to in this application are specifically incorporated by reference herein. In case of conflict, the present specification, including its specific definitions, will control.

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The definition of each expression, e.g., alkyl, m, n, and the like, when it occurs more than once in any structure, is intended to be independent of its definition elsewhere in the same structure.

Certain compounds may exist in particular geometric or stereoisomeric forms. In addition, polymers of the invention may also be optically active. The invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

For purposes of this invention, the chemical elements are identified in accordance with the Periodic Table of the Elements, CAS version, Handbook of Chemistry and Physics, 67th Ed., 1986-87, inside cover.

The term “mixing” refers to any method of contacting one component of a mixture with another component of a mixture, including agitating, blending, combining, contacting, milling, shaking, sonicating, spraying, stirring, and vortexing.

An “alkyl” group is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters),—CF₃, —CN and the like. Exemplary substituted alkyls are described below. Cycloalkyls can be further substituted with alkyls, alkenyls, alkoxys, alkylthios, aminoalkyls, carbonyl-substituted alkyls,—CF₃,—CN, and the like.

The term “C_{x-y},” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The terms “halo” and “halogen” as used herein means halogen and includes chloro, fluoro, bromo, and iodo.

The term “heteroatom” as used herein means an atom of any element other than carbon or hydrogen. Preferred heteroatoms are nitrogen, oxygen, and sulfur.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “silyl” refers to a silicon moiety with three hydrocarbyl moieties attached thereto.

The term “silyloxy” refers to an oxygen moiety with a silyl attached thereto.

The term “activating ester moiety”, as used herein, refers to any ester group, i.e. C(O)O—Y, wherein O—Y is an activating group which makes the carbonyl carbon highly susceptible toward nucleophilic attack. The activating group may be, but is not limited to,

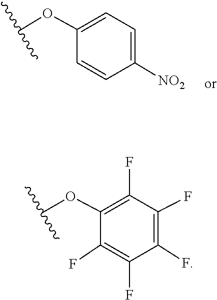 [SMILES: *Oc1ccc([N+](=O)[O-])cc1 ; *Oc1c(F)c(F)c(F)c(F)c1F]

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base polypeptides disclosed herein. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of polypeptides are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of polypeptides for laboratory use, or for subsequent conversion to a pharmaceutically acceptable acid addition salt.

The term “pharmaceutically acceptable basic addition salt” as used herein means any non-toxic organic or inorganic base addition salt of any acid polypeptides represented by Formula I or II. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The invention now being generally described, it will be more readily understood by reference to the following examples which are included merely for purposes of illustration of certain aspects and embodiments of the present invention, and are not intended to limit the invention.

### Examples

The invention is further described in the following examples, which do not limit the scope of the invention described in the claims.

### Example 1: Preliminary Synthesis of Enantiopure (−)-Cannabidiol (CBD)

Enantiopure (−)-Cannabidiol (CBD) was prepared starting from commercially available (R)-limonene.

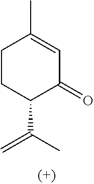

1. (S)-3-Methyl-6-(prop-1-en-2-yl)cyclohex-2-enone; common name (+)-(S)-isopiperitenone (Dethe, D. H. et al. 2015). A flame-dried, 100 mL round bottom flask was charged with 40 mL of tert-butanol by syringe. With vigorous magnetic stirring, 16.0 g (160 mmol, 4.0 equiv) of powdered chromium (VI) oxide was added in portions. Initial dissolution of the oxidant causes a bright orange solution to develop, but with continued stirring the solution becomes deep maroon in color and slight warming is observed. After 10 minutes at 25° C., the solution was cooled to 0° C., diluted with 40 mL of chloroform, and transferred to a 250 mL separatory funnel. Ice-cold deionized water (50 mL) was added, and the mixture was agitated before allowing the phases to separate. The lower organic layer (bright red) was removed and the upper aqueous layer (light brown) was back-extracted twice with 20 mL portions of chloroform. The pooled organic layers were dried over sodium sulfate and vacuum filtered through cotton in a coarse porosity fritted funnel directly into a flame-dried 250 mL round bottom receiving flask. To the resulting deep red filtrate was added 6.50 mL (40.2 mmol, 1.0 equiv) of (R)-limonene. With continuous stirring, the vessel was equipped with a heating mantle and the solution brought to a gentle reflux. After 1 hour, the reaction mixture had become turbid and dark brown in color. The heat source was removed and stirring continued overnight at 25° C., at which point TLC analysis confirmed the absence of starting material and production of two regioisomeric enones. The mixture was directly filtered to remove insoluble chromium (IV) deposits, and the resulting brown filtrate was concentrated on a rotary evaporator in the presence of 10 grams of added silica gel. Purification of the residue by column chromatography with 10:1 petroleum ether: diethyl ether as eluent and a KMnO₄ stain for spot visualization furnished(S)-carvone as a viscous oil (R_{f}=0.55) as the major isomer (1.99 g, 33%) and isopiperitenone as a yellow liquid (R_{f}=0.40, 1.03 g, 17%). [α]²⁸D=+27.88 (c 0.54, CHCl₃); IR (neat, cm⁻¹) 3076, 2927, 1667, 1435, 1379, 1201, 1087, 891; 1H NMR (CDCl₃, 400 MHZ) δ 5.89 (d, J=1.5 Hz, 1H), 4.94 (d, J=1.5 Hz, 1H), 4.75 (d, J=1.5 Hz, 1H), 2.94 (dd, J=10.5, 5.4 Hz, 1H), 2.35-2.31 (m, 2H), 2.11-1.98 (m, 2H), 1.94 (s, 3H), 1.74 (d, J=1.5 Hz, 3H); ¹³C NMR (CDCl₃, 100 MHZ) δ 199.5, 162.0; 143.4, 126.8, 113.6, 53.9, 30.4, 27.7, 24.3, 20.7; HRMS (ESI+) Calcd. for C₁₀H₁₄O [M]⁺: 150.1045; Found 150.1043.

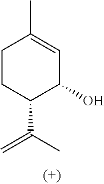

2. (1S, 6S)-3-methyl-6-(prop-1-en-2-yl)cyclohex-2-enol; common name (±)-cis-isopiperitenol. A flame-dried, 100 mL round bottom flask was charged with 808 mg (5.38 mmol, 1.0 equiv) of neat(S)-isopiperitenone and 15 mL of anhydrous methanol as solvent (0.36 M concentration). With magnetic stirring at 25° C., 996 mg (2.67 mmol, 0.50 equiv) of cerium (III) chloride hepta-hydrate was added as a colorless, blocky solid in one portion, which slowly dissolved to give a homogeneous solution. After 10 minutes, the solution was cooled to −10° C. (methanol/ice bath) and pulverized sodium borohydride (204 mg, 5.39 mmol, 1.0 equiv) was added in two portions causing immediate hydrogen gas evolution in the form of gentle bubbling. The resulting cloudy mixture was stirred for 1 hour and the cold bath removed. After diluting with 40 mL of deionized water, the reaction mixture was transferred to a 125 mL separatory funnel and extracted 3× with 30 mL of dichloromethane. Alternative extraction solvents (diethyl ether and ethyl acetate) were tested but led to an emulsion. Some heterogeneity is seen with dichloromethane, but the bilayer returns upon 10-15 min of standing. The combined organic layers were dried over MgSO₄, filtered through cotton, and concentrated to furnish 750 mg of an orange liquid. This material consists mostly of 1,4-reduction product (R_{f}=0.62) due to steric hindrance associated with 1,2-reduction. Purification by flash silica gel chromatography with 3:1 petroleum ether: diethyl ether as eluent and CAM staining gave 303 mg of (±)-cis-isopiperitenol (R_{f}=0.25, 37% yield) as a colorless oil. A trace of trans diastereomer (R_{f}=0.26, <4% yield) was observed to elute just ahead of product, reflecting greater than 10:1 stereoselectivity when the hydride agent is added at −10° C. Spectral characteristics for this substance were identical in all respects to material of 94% ee prepared previously by a multi-step asymmetric synthesis (Tomooka, K. et al. 2005).

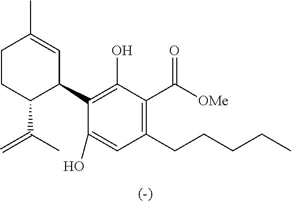 [SMILES: C=C(C)[C@@H]1CCC(C)=C[C@H]1c1c(O)cc(CCCCC)c(C(=O)OC)c1O]

3. (−)-Cannabidiol acid methyl ester, common name methyl cannibidiolate. A flame-dried 1 dram vial was charged in air with 23.8 mg of methyl 2,4-dihydroxy-6-pentylbenzoate (0.100 mmol, 1.1 equiv) and 7.2 mg of camphorsulfonic acid (0.031 mmole, 0.34 equiv) as solids and then 0.6 mL of anhydrous dichloromethane was added as solvent after purging the vial with nitrogen gas. To the resulting clear solution stirring at 0° C. was added 13.8 mg (0.0907 mmol, 0.91 equiv) of (±)-cis-isopiperitenol in 0.4 mL of dichloromethane dropwise over 1 hour. The reaction mixture was stirred for an additional hour at 0° C., the ice bath removed, and nitrogen gas purge line (and vent needle) was used to concentrate the reaction mixture to a neat oil at 25° C. The vial contents were then resuspended in 1.0 mL of diethyl ether, 1.0 mL of cold saturated sodium bicarbonate was added to quench the acid catalyst, and after agitation by stirring the organic layer was drawn out by Pasteur pipette. The aqueous layer was washed 2× with 1.0 mL of diethyl ether and the extracts pooled, dried over MgSO₄, filtered through cotton, and concentrated to an oily residue. Purification by silica gel chromatography in 9:1 petroleum ether: diethyl ether furnished the title compound as a yellow oil (21.1 mg, 60%, R_{f}=0.55, UV active spot or dark blue to CAM stain). ¹H NMR (CDCl₃, 500 MHz) δ 12.00 (s, 1H), 6.51 (s, 1H), 6.21 (s, 1H), 5.55 (s, 1H), 4.52 (s, 1H), 4.37 (s, 1H), 4.10 (br s, 1H), 3.90 (s, 3H), 2.87-2.81 (m, 1H), 2.76-2.69 (m, 1H), 2.71-2.65 (m, 1H), 2.42-2.32 (m, 1H), 2.34-2.27 (m, 1H), 2.26-2.17 (m, 1H), 2.12-2.06 (m, 1H), 1.79 (s, 3H), 1.71 (s, 3H), 1.54-1.46 (m, 2H), 1.35-1.27 (m, 2H), 1.27-1.23 (m, 2H), 0.89 (t, J=6.5 Hz, 3H).

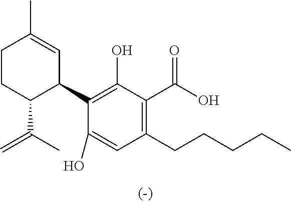 [SMILES: C=C(C)[C@@H]1CCC(C)=C[C@H]1c1c(O)cc(CCCCC)c(C(=O)O)c1O]

### 4. (−)-Cannabidiol (CBD).

Basic hydrolysis with LiOH (1 eq.) in THE provides the free acid followed by thermal decarboxylation to provide (−)-Cannabidiol (CBD).

### Example 2: Preliminary Synthesis of Racemic (±)-Cannabidiol (CBD)

The methods described in Example 1 were adapted to provide racemic (±)-Cannabidiol (CBD). In particular, the following method was used to provide (±)-cis-isopiperitenol, which is then subjected to Steps 3 and 4 of Example 1.

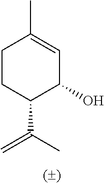

1. Rac-(±)-cis-3-methyl-6-(prop-1-en-2-yl)cyclohex-2-enol; common name cis-isopiperitenol. A 1.75 gram sample of commercial citral (≥96%, mixture of trans (geranial) and cis (neral), was filtered through a column (5 cm diameter×10 cm high) of silica gel slurry-packed in benzene containing 3% diethyl ether (R_{f}=0.54; UV active or yellow spot under KMnO₄ staining). This flash chromatography step removes a yellow-colored impurity that elutes with the solvent front, and it also serves to rigorously dry the substrate due to azeotropic removal of any traces of water. Concentration of pure citral fractions on a rotary evaporator, backfilling of the apparatus with an argon ballon, and further degassing under high vacuum and with magnetic stirring of the neat oil furnished 1.52 g (10.0 mmol, 1.0 equiv) of citral that was completely colorless. The starting material was dissolved in 10.0 mL of anhydrous dichloromethane, and a 12 mL-capacity plastic syringe was charged with the solution. With degassing under positive nitrogen pressure, a second disposable gas-tight syringe was loaded with commercial dimethylaluminum chloride (10.1 mL of 1.0 M in hexanes, 10.1 mmol, 1.01 equiv). Lastly, a separate flame-dried, 24/40 jointed, 100 mL round bottom flask with a side-arm stopcock and hose connector (for nitrogen pressure) was charged with 10.0 mL of dichloromethane and cooled to −10° C. in an ice/methanol bath. At the same addition rates using two syringe pumps, the substrate and promoter solutions were added dropwise to the chilled flask over three hours. The exit needle for the promoter solution was placed below the surface of solvent to minimize smoking or cloudiness in the headspace of the reaction flask. The role of slow addition is to mimic high dilution and minimize intermolecular reactions. Over the course of the addition, the reaction mixture turns light orange and eventually maroon in color. Stirring was continued with slow warming to room temperature for one hour. The mixture was then quenched at 0° C. by the sequential addition of 1.0 mL of deionized water, 1.0 mL of 4 M aqueous sodium hydroxide, and an additional 3.0 mL of water. These additions, which mirror a standard workup for a lithium aluminum hydride reaction, cause methane gas evolution, bleaching of the orange color in the reaction mixture, and formation of a milky precipitate from which the yellow supernatant was easily isolated by decantation. Thus, filtration of the mixture through a pad of cotton, washing the aluminum (III) hydroxide precipitate with extra dichloromethane, and concentration afforded a bronze oil consisting primarily of unreacted citral (R=0.71) and product (R_{f}=0.38 in 2:1 heptane: diethyl ether with either KMnO₄ or ceric ammonium molybdate (CAM) TLC staining. Purification was achieved by chromatography over silica gel in the same solvent system, providing 228 mg (15% yield) of a colorless oil. IR (neat, cm⁻¹) 3352, 3088, 2936, 1676, 1649, 1450, 1379, 1245, 1210, 1176, 1156, 1114, 1069, 1025, 957, 915, 886, 849, 816, 723, 681 cm⁻¹; ¹H NMR (CDCl₃, 400 MHZ) δ 5.67 (m, 1H), 4.99 (s, 1H), 4.81 (s, 1H), 4.12 (br s, 1H), 2.17-2.04 (m, 3H), 1.83 (s, 3H), 1.80-1.74 (m, 1H), 1.72 (s, 3H), 1.65-1.55 (m, 1H), 1.48 (br s, 1H); ¹³C NMR (CDCl₃, 100 MHz) δ 146.7, 139.9, 122.5, 111.8, 63.9, 46.2, 31.3, 23.5, 22.7, 21.0; HRMS (ESI+) Calcd. for C₁₀H₁₆O [M]⁺: 152.1201; Found 150.1209.

### Example 3: Preliminary Synthesis of Enantiopure (−)-Delta-9-Tetrahydrocannabinol (Δ⁹-THC)

Enantiopure (−)-delta-9-Tetrahydrocannabinol (Δ⁹-THC) was prepared starting from commercially available (R)-limonene.

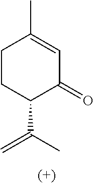

1. (S)-3-Methyl-6-(prop-1-en-2-yl)cyclohex-2-enone; common name (+)-(S)-isopiperitenone (Dethe, D. H. et al. 2015). A flame-dried, 100 mL round bottom flask was charged with 40 mL of tert-butanol by syringe. With vigorous magnetic stirring, 16.0 g (160 mmol, 4.0 equiv) of powdered chromium (VI) oxide was added in portions. Initial dissolution of the oxidant causes a bright orange solution to develop, but with continued stirring the solution becomes deep maroon in color and slight warming is observed. After 10 minutes at 25° C., the solution was cooled to 0° C., diluted with 40 mL of chloroform, and transferred to a 250 mL separatory funnel. Ice-cold deionized water (50 mL) was added, and the mixture was agitated before allowing the phases to separate. The lower organic layer (bright red) was removed and the upper aqueous layer (light brown) was back-extracted twice with 20 mL portions of chloroform. The pooled organic layers were dried over sodium sulfate and vacuum filtered through cotton in a coarse porosity fritted funnel directly into a flame-dried 250 mL round bottom receiving flask. To the resulting deep red filtrate was added 6.50 mL (40.2 mmol, 1.0 equiv) of (R)-limonene. With continuous stirring, the vessel was equipped with a heating mantle and the solution brought to a gentle reflux. After 1 hour, the reaction mixture had become turbid and dark brown in color. The heat source was removed and stirring continued overnight at 25° C., at which point TLC analysis confirmed the absence of starting material and production of two regioisomeric enones. The mixture was directly filtered to remove insoluble chromium (IV) deposits, and the resulting brown filtrate was concentrated on a rotary evaporator in the presence of 10 grams of added silica gel. Purification of the residue by column chromatography with 10:1 petroleum ether: diethyl ether as eluent and a KMnO₄ stain for spot visualization furnished(S)-carvone as a viscous oil (R=0.55) as the major isomer (1.99 g, 33%) and isopiperitenone as a yellow liquid (R=0.40, 1.03 g, 17%). [α]²⁸D)=+27.88 (c 0.54, CHCl₃); IR (neat, cm⁻¹) 3076, 2927, 1667, 1435, 1379, 1201, 1087, 891; ¹H NMR (CDCl₃, 400 MHZ) δ 5.89 (d, J=1.5 Hz, 1H), 4.94 (d, J=1.5 Hz, 1H), 4.75 (d, J=1.5 Hz, 1H), 2.94 (dd, J=10.5, 5.4 Hz, 1H), 2.35-2.31 (m, 2H), 2.11-1.98 (m, 2H), 1.94 (s, 3H), 1.74 (d, J=1.5 Hz, 3H); ¹³C NMR (CDCl₃, 100 MHZ) 8 199.5, 162.0; 143.4, 126.8, 113.6, 53.9, 30.4, 27.7, 24.3, 20.7; HRMS (ESI+) Calcd. for C₁₀H₁₄O [M]⁺: 150.1045; Found 150.1043.

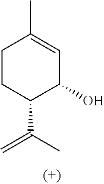

2. (1S, 6S)-3-methyl-6-(prop-1-en-2-yl)cyclohex-2-enol; common name (±)-cis-isopiperitenol. A flame-dried, 100 mL round bottom flask was charged with 808 mg (5.38 mmol, 1.0 equiv) of neat(S)-isopiperitenone and 15 mL of anhydrous methanol as solvent (0.36 M concentration). With magnetic stirring at 25° C., 996 mg (2.67 mmol, 0.50 equiv) of cerium (III) chloride hepta-hydrate was added as a colorless, blocky solid in one portion, which slowly dissolved to give a homogeneous solution. After 10 minutes, the solution was cooled to −10° C. (methanol/ice bath) and pulverized sodium borohydride (204 mg, 5.39 mmol, 1.0 equiv) was added in two portions causing immediate hydrogen gas evolution in the form of gentle bubbling. The resulting cloudy mixture was stirred for 1 hour and the cold bath removed. After diluting with 40 mL of DI water, the reaction mixture was transferred to a 125 mL separatory funnel and extracted 3× with 30 mL of dichloromethane. Alternative extraction solvents (diethyl ether and ethyl acetate) were tested but led to an emulsion. Some heterogeneity is seen with dichloromethane, but the bilayer returns upon 10-15 min of standing. The combined organic layers were dried over MgSO₄, filtered through cotton, and concentrated to furnish 750 mg of an orange liquid. This material consists mostly of 1,4-reduction product (R_{f}=0.62) due to steric hindrance associated with 1,2-reduction. Purification by flash silica gel chromatography with 3:1 petroleum ether: diethyl ether as eluent and CAM staining gave 303 mg of (±)-cis-isopiperitenol (R_{f}=0.25, 37% yield) as a colorless oil. A trace of trans diastereomer (R_{f}=0.26, <4% yield) was observed to elute just ahead of product, reflecting greater than 10:1 stereoselectivity when the hydride agent is added at −10° C. Spectral characteristics for this substance were identical in all respects to material of 94% ee prepared previously by a multi-step asymmetric synthesis (Tomooka, K. et al. 2005).

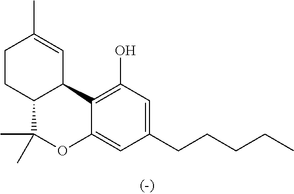 [SMILES: CCCCCc1cc(O)c2c(c1)OC(C)(C)[C@@H]1CCC(C)=C[C@@H]21]

3. delta-9-Tetrahydrocannabinol (Δ⁹-THC), International Nonproprietary Name Dronabinol. para-Toluenesulfonic acid monohydrate was recrystallized (as thin needles) from boiling chloroform, washed with hexane, and dried in vacuo prior to usage. A flame-dried 10 mL round bottom flask was charged with 20.0 mg of olivetol (0.111 mmol, 1.0 equiv) and 6.8 mg of p-TsOH (0.039 mmol, 0.35 equiv) as solids and 1.7 mL of anhydrous dichloromethane under positive pressure of nitrogen. To the resulting clear solution stirring at 25° C. was added 18.6 mg (0.122 mmol, 1.1 equiv) of (±)-cis-isopiperitenol (Cardillo, B. et al. 1973) in 0.5 mL of dichloromethane dropwise over 1 hour. TLC analysis of the mixture after 2 hours of reaction showed only traces amounts of the reactants and a prominent non-polar spot (R_{f}=>0.90 in 3:1 hexane: diethyl ether). The reaction was quenched with 2.0 mL of saturated sodium bicarbonate and the organic layer removed. The water layer was washed with 2×1.0 mL dichloromethane and the extracts combined, dried over MgSO₄, filtered through cotton, and concentrated to an oily residue. Purification by passage through silica gel in 50:1 petroleum ether:ethyl acetate delivered 12.7 mg (33%) of a clean 3:1 mixture of 49-THC and Δ⁸-THC (Hively, R. L. et al. 1966) as the first compounds to elute. Separation by HPLC has been reported under the following conditions: Sunfire C18 5μ, 4.6×150 mm column, 1 mL/min flow, gradient elution 80% water in acetonitrile to 100% acetonitrile containing 0.5-1% TFA. Spectroscopic data collected for synthetic Δ⁹-THC match that of both the natural product (Gaoni, Y. et al. 1964) and racemic material prepared according to an early biomimetic synthesis (Taylor, E. C. et al. 1966).

### Example 4: Preliminary Synthesis of Racemic (±)-Delta-9-Tetrahydrocannabinol (49-THC)

The methods described in Example 3 were adapted to provide racemic (±)-delta-9-Tetrahydrocannabinol (Δ⁹-THC). In particular, the following method was used to provide (±)-cis-isopiperitenol, which is then subjected to Step 3 of Example 3.

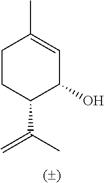

1. Rac-(±)-cis-3-methyl-6-(prop-1-en-2-yl)cyclohex-2-enol; common name cis-isopiperitenol. A 1.75 gram sample of commercial citral (>96%, mixture of trans (geranial) and cis (neral), was filtered through a column (5 cm diameter×10 cm high) of silica gel slurry-packed in benzene containing 3% diethyl ether (R_{f}=0.54; UV active or yellow spot under KMnO₄ staining). This flash chromatography step removes a yellow-colored impurity that elutes with the solvent front, and it also serves to rigorously dry the substrate due to azeotropic removal of any traces of water. Concentration of pure citral fractions on a rotary evaporator, backfilling of the apparatus with an argon ballon, and further degassing under high vacuum and with magnetic stirring of the neat oil furnished 1.52 g (10.0 mmol, 1.0 equiv) of citral that was completely colorless. The starting material was dissolved in 10.0 mL of anhydrous dichloromethane, and a 12 mL-capacity plastic syringe was charged with the solution. With degassing under positive nitrogen pressure, a second disposable gas-tight syringe was loaded with commercial dimethylaluminum chloride (10.1 mL of 1.0 M in hexanes, 10.1 mmol, 1.01 equiv). Lastly, a separate flame-dried, 24/40 jointed, 100 mL round bottom flask with a side-arm stopcock and hose connector (for nitrogen pressure) was charged with 10.0 mL of dichloromethane and cooled to −10° C. in an ice/methanol bath. At the same addition rates using two syringe pumps, the substrate and promoter solutions were added dropwise to the chilled flask over three hours. The exit needle for the promoter solution was placed below the surface of solvent to minimize smoking or cloudiness in the headspace of the reaction flask. The role of slow addition is to mimic high dilution and minimize intermolecular reactions. Over the course of the addition, the reaction mixture turns light orange and eventually maroon in color. Stirring was continued with slow warming to room temperature for one hour. The mixture was then quenched at 0° C. by the sequential addition of 1.0 mL of deionized water, 1.0 mL of 4 M aqueous sodium hydroxide, and an additional 3.0 mL of water. These additions, which mirror a standard workup for a lithium aluminum hydride reaction, cause methane gas evolution, bleaching of the orange color in the reaction mixture, and formation of a milky precipitate from which the yellow supernatant was easily isolated by decantation. Thus, filtration of the mixture through a pad of cotton, washing of the aluminum (III) hydroxide precipitate with extra dichloromethane, and concentration afforded a bronze oil consisting primarily of unreacted citral (R_{f}=0.71) and product (R_{f}=0.38 in 2:1 heptane: diethyl ether with either KMnO₄ or ceric ammonium molybdate (CAM) TLC staining. Purification was achieved by chromatography over silica gel in the same solvent system, providing 228 mg (15% yield) of a colorless oil. IR (neat, cm⁻¹) 3352, 3088, 2936, 1676, 1649, 1450, 1379, 1245, 1210, 1176, 1156, 1114, 1069, 1025, 957, 915, 886, 849, 816, 723, 681 cm⁻¹; ¹H NMR (CDCl₃, 400 MHZ) δ 5.67 (m, 1H), 4.99 (s, 1H), 4.81 (s, 1H), 4.12 (br s, 1H), 2.17-2.04 (m, 3H), 1.83 (s, 3H), 1.80-1.74 (m, 1H), 1.72 (s, 3H), 1.65-1.55 (m, 1H), 1.48 (br s, 1H); ¹³C NMR (CDCl₃, 100 MHZ) δ 146.7, 139.9, 122.5, 111.8, 63.9, 46.2, 31.3, 23.5, 22.7, 21.0; HRMS (ESI+) Calcd. for C₁₀H₁₆O [M]⁺: 152.1201; Found 150.1209.

### Example 5: Preliminary Synthesis of Cannabigerolic Acid (CBGA) and Cannabigerol (CBG)

Cannabigerolic acid (CBGA) and Cannabigerol (CBG) were prepared starting from commercially available geraniol and synthetic methyl olivetolate (see below, Example 6).

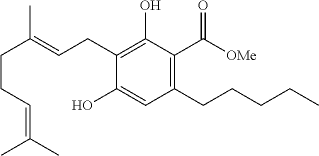 [SMILES: CCCCCc1cc(O)c(C/C=C(\C)CCC=C(C)C)c(O)c1C(=O)OC]

1. Methyl (E)-3-(3,7-dimethylocta-2,6-dien-1-yl)-2,4-dihydroxy-6-pentylbenzoate; cannabigerolic acid methyl ester, CBGA methyl ester. A flame-dried 10 mL round bottom flask was charged in air with 125 mg of methyl 2,4-dihydroxy-6-pentylbenzoate (0.525 mmol, 1.0 equiv) and recrystallized para-toluenesulfonic acid monohydrate (thin needles from chloroform, 20 mg, 0.11 mmol, 0.21 equiv) as solids and then 2.25 mL of anhydrous dichloromethane was added as solvent after purging the vial with nitrogen gas. To the resulting light yellow solution stirring at 0° C. was added 80.9 mg (0.524 mmol, 0.998 equiv) of geraniol in 3.0 mL of dichloromethane dropwise over 15 minutes. The reaction mixture was stirred for an additional hour at 0° C., the ice bath removed, and nitrogen gas purge line (and vent needle) was used to concentrate the reaction mixture to a neat oil at 25° C. The contents of the flask were suspended in 20 mL of diethyl ether and the reaction quenched by pouring it over 20 mL of cold aqueous saturated sodium bicarbonate. The aqueous layer was washed two times with 5 mL of diethyl ether and the extracts pooled, dried over MgSO₄, filtered through cotton, and concentrated to an oily residue. Purification by silica gel chromatography in 3:1 petroleum ether:ethyl acetate furnished the title compound as an off-white solid (118 mg, 60%, R_{f}=0.30, UV active spot or dark blue to CAM stain). Spectroscopic data for this material was in agreement with that of a literature report (Mechoulam, R. et al. 1965).

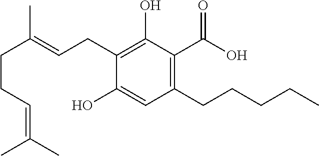 [SMILES: CCCCCc1cc(O)c(C/C=C(\C)CCC=C(C)C)c(O)c1C(=O)O]

Basic hydrolysis with LiOH in THE provides the free acid (CBGA) (Mechoulam, R. et al. 1965).

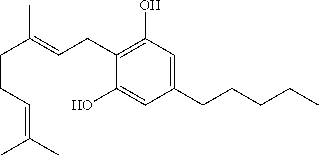 [SMILES: CCCCCc1cc(O)c(C/C=C(\C)CCC=C(C)C)c(O)c1]

Hydrolysis with thermal decarboxylation furnishes cannabigerol (CBG) (Mechoulam, R. et al. 1965).

### Example 6: Synthesis of Methyl Olivetolate

Methyl olivetolate (Compound (III) where R₁ is pentyl and R₄ is methyl) was prepared by two different methods.

### Multi-Step Method

1. Ethyl 3-oxo-octanoate. A 250 mL round bottom flask equipped with a teflon-coated spin bar and a wide 24/40 ground glass joint was flame-dried under vacuum and backfilled with nitrogen. Anhydrous tin (II) chloride (1.5 g, 7.9 mmol, 0.099 equiv) was pulverized in a mortar and pestle, weighed in air, and added to the flask, at which point the vacuum drying procedure was repeated with the solid present and gently agitated by magnetic stirring. Under positive nitrogen pressure, the rigorously dry catalyst was suspended in 110 mL of dichloromethane obtained from a solvent purification system and with stirring, 12.5 g of ethyl diazoacetate (80.1 mmol, 1.00 equiv) was added by syringe as a solution in 50 mL of dichloromethane (Womack, E. B. and Nelson, A. B., 1944). To the resulting cloudy and yellow mixture at ambient temperature, 9.8 mL of distilled hexanal (80 mmol, 1.0 equiv) was added dropwise over 15 minutes and vigorous nitrogen evolution was observed. After 1 hour of stirring, bubbling had ceased and the mixture had turned homogeneous. The reaction was transferred to a 500 mL separatory funnel containing 100 mL of saturated sodium chloride and extracted with 2× 200 mL of diethyl ether. The organic layers were pooled, dried over magnesium sulfate, filtered, and concentrated by rotary evaporation. The resulting liquid was passed through a plug of cotton and all remaining volatiles were removed in vacuo, providing 15.0 g (quant. yield) of a colorless, translucent oil that proved to be a single spot by TLC analysis (R_{f}=0.55 in 4:1 petroleum ether: diethyl ether with KMnO₄ staining). IR (neat, cm⁻¹) 2933, 1741, 1716, 1647, 1466, 1411, 1368, 1308, 1233, 1152, 1096, 1029, 847 cm⁻¹; ¹H NMR (CDCl₃, 500 MHZ) 8 4.19 (q, J=7.3 Hz, 2H), 3.43 (s, 2H), 2.53 (t, J=7.3 Hz, 1H), 1.60 (quintet, J=6.9 Hz, 2H), 1.33-1.24 (m, 7H), 0.88 (t, J=6.9 Hz, 3H); ¹³C NMR (CDCl₃, 125 MHZ) δ 203.2, 167.4, 61.5, 49.5, 43.2, 31.3, 23.3, 22.5, 14.2, 14.0; HRMS (ESI+) Calcd. for C₁₀H₁₈O₃Na [M+Na]⁺: 209.1154; Found 209.1155.

2. Ethyl 2-(2-pentyl-1,3-dioxolan-2-yl)acetate. To a solution of 5.70 g (30.6 mmol, 1.0 equiv) of ethyl 3-oxo octanoate in 2-ethyl-2-methyl-1,3-dioxolane (9.0 mL, 72 mmol, 2.4 equiv) in a flame-dried 100 mL round bottom flask was added 1.75 g of para-toluene sulfonic acid hydrate (9.20 mmol, 0.30 equiv) in one portion as a solid. The acid catalyst quickly dissolved, and the colorless solution was stirred for 12 hours at 25° C. The reaction mixture was quenched with 60 mL of saturated (aq.) sodium bicarbonate, transferred to a 125 mL separatory funnel, and washed three times with 50 mL diethyl ether. The combined organic layers were dried over MgSO₄ and concentrated to afford the crude ethylene ketal as a yellow oil. Purification was achieved by flash chromatography over silica gel in 4:1 cyclohexane: diethyl ether as eluent and with KMnO₄ as a stain. Product was obtained as a colorless, free-flowing oil (R_{f}=0.50, 5.64 g, 80%) along with a minor amount of recovered starting material (R_{f}=0.38, 210 mg, 3.7%). IR (neat, cm⁻¹) 2933, 1735, 1628, 1466, 1369, 1199, 1095, 1031, 949, 855 cm⁻¹; ¹H NMR (CDCl₃, 500 MHZ) δ 4.15 (q, J=7.3 Hz, 2H), 4.01-3.94 (m, 4H), 2.64 (s, 2H), 1.79 (ddd, J=5.4, 2.9, 1.0 Hz, 2H), 1.42-1.36 (m, 2H), 1.33-1.27 (m, 4H), 1.26 (t, J=7.3 Hz, 3H), 0.88 (t, J=7.3 Hz, 3H); ¹³C NMR (CDCl₃, 125 MHZ) δ 169.8, 109.6, 65.2, 60.6, 42.7, 37.9, 32.0, 23.3, 22.7, 14.3, 14.2; HRMS (ESI+) Calcd. for C₁₂H2204Na [M+Na]⁺: 253.1416; Found 253.1413. Anal Calcd for C₁₂H₂₂O₄: C, 62.58; H, 9.63. Found: C, 63.35; H, 9.55.

3. 2-(2-pentyl-1,3-dioxolan-2-yl) acetic acid. A 200 mL round bottom flask was charged with 2.31 g (10.0 mmol, 1.0 equiv) of ethyl 2-(2-pentyl-1,3-dioxolan-2-yl)acetate in air, and the oil was dissolved in 30 mL of absolute ethanol. With magnetic stirring at 25° C., 20.5 mL of a 0.5 N standardized sodium hydroxide solution (10.3 mmol, 1.03 equiv) was added by syringe, causing the solution to become slightly turbid and yellow in color. The flask was placed into an oil bath preheated to 80° C., and the reaction mixture was refluxed for 2 hours, at which point the mixture became homogeneous. Stirring was continued at 25° C. for 12 hours, and the solution was briefly concentrated by rotary evaporation to remove ethanol. The reaction mixture was then quenched at 0° C. by the addition of 15 mL of saturated (aq.) ammonium chloride, diluted with 20 mL of cold diethyl ether, and transferred to a 125 mL separatory funnel to permit the layers to separate. The organic layer was isolated, and the aqueous layer was treated with an additional 30 mL of ammonium chloride, causing visible cloudiness. The mixture was again extracted with 30 mL of cold ether. A routine litmus paper test showed the aqueous layer to be at pH 6-7. Therefore, the acidification was repeated, but this time with formic acid (300 μL) so as to reach a final pH of 5. A third 30 mL ether extract was collected and all organic layers were pooled, dried over MgSO₄, and filtered through cotton. Concentration delivered a colorless oil that was purified by filtration through a short but wide pad of silica gel in 4:1:1 diethyl ether: dichloromethane: petroleum ether to remove a trace of starting material (R_{f}=0.80). Gradient elution with 100% diethyl ether and KMnO₄ visualization delivered the acid product (R_{f}=0.45 in starting solvent) as a colorless oil that was stored under nitrogen at −20° C. (1.54 g, 76%). ¹H NMR (CDCl₃, 500 MHz) δ 4.04-3.98 (m, 4H), 2.70 (s, 3H), 1.78 (ddd, J=5.4, 2.9, 1.0 Hz, 2H), 1.42-1.36 (m, 2H), 1.32-1.23 (m, 4H), 0.88 (t, J=6.8 Hz, 3H); ¹³C NMR (CDCl₃, 125 MHZ) δ 175.3, 109.5, 65.2, 42.4, 37.7, 31.9, 23.3, 22.7, 14.2, 14.1. HRMS (ESI+) Calcd. for C₁₀H₁₈O₄Na [M+Na]⁺: 225.1097; Found 225.1099.

4. Methyl 2,4-dihydroxy-6-pentylbenzoate. A flame-dried 100 mL round bottom flask equipped with a teflon-coated spin bar was charged with 1.54 g (7.61 mmol, 1.0 equiv) of 2-(2-pentyl-1,3-dioxolan-2-yl) acetic acid followed by 30 mL of dry benzene. After stirring the solution at 25° C. for 5 minutes, oxalyl chloride (0.64 mL, 7.6 mmol, 0.99 equiv) was added dropwise from a syringe under a positive pressure of nitrogen. After placing a heating mantle under the vessel, the reaction mixture was brought to reflux for 2 hours with continuous stirring. The flask was then cooled to 25° C., the stir bar was removed, and the benzene was removed by rotary evaporation. Heating was avoided during the removal of solvent, as the acid chloride product decomposes upon standing at room temperature. The resulting light yellow oil, obtained in >98% yield based on mass difference, was used immediately and without purification in the subsequent annulation.

Thus, a second flame-dried 100 mL round bottom flask equipped with a teflon-coated spin bar was charged with 1.96 g (7.52 mmol, 1.0 equiv) of (E)-1,3-bis (trimethylsiloxy)-1-methoxybuta-diene prepared by the procedure of Chan and Brownbridge (Chan, T-H., Brownbridge, P. A., 1980).

The 2-(2-pentyl-1,3-dioxolan-2-yl) acetyl chloride (1.66 g, 7.52 mmol, 1.0 equiv), as prepared above, was dissolved in 30 mL of dry dichloromethane and added to the flask containing the bis (silyl enol ether) through a steel cannula under a positive nitrogen pressure. With continuous stirring, the resulting yellow-orange mixture was treated with a solution of titanium tetrachloride (1.65 mL, 15.0 mmol, 2.0 equiv) in 8.0 mL of dichloromethane (0.20 M final concentration). After 24 hours of stirring at 25° C., the reaction mixture was poured into 50 mL of cold saturated (aq.) sodium bicarbonate in a 125 mL separatory funnel and extracted three times with 30 mL of diethyl ether. The combined organic layers were dried over MgSO₄, filtered, and concentrated by rotary evaporation. The crude product was purified by flash chromatography over silica gel with 4:1 benzene: diethyl ether (R_{f}=0.35) as eluent and a KMnO+stain for spot visualization. Concentration of pure product fractions resulted in spontaneous deposition of the methyl olivetolate as a yellow solid (986 mg, 55%). Mp=77-78° C.; IR (neat, cm⁻¹) 3600, 3400 (phenolic OH bands), 1660 (ester); ¹H NMR (CDCl₃, 500 MHz) δ 11.68 (s, 1H), 6.28 (d, J=2.9 Hz, 1H), 6.23 (d, J=2.5 Hz, 1H), 5.26 (br s, 1H), 3.92 (s, 3H), 2.83 (t, J=7.8 Hz, 2H), 1.56-1.49 (m, 2H), 1.36-1.30 (m, 4H), 0.90 (t, J=6.8 Hz, 3H); 13C NMR (CDCl₃, 125 MHz) δ 172.1, 165.4, 160.4, 149.1, 110.9, 104.8, 101.5, 52.1, 37.0, 32.2, 31.6, 22.7, 14.2. HRMS (ESI+) Calcd. for C₁₃H₁₉O₄Na [M+H]⁺: 239.1283; Found 239.1279.

### One-Step Method

1. Methyl 2,4-dihydroxy-6-pentylbenzoate. Methyl 6-n-pentyl-2-hydroxy-4-oxo-cyclohex-2-ene-1-carboxylate (2.62 g, 10.9 mmol), prepared as a white flaky solid by a literature procedure (Focella, A. et al. 1977), was added to a 100 mL round bottom flask containing a teflon-coated spin bar and dissolved in 10 mL of anhydrous DMF at 25° C., giving a viscous yellow solution. A separate flame-dried 50 mL round bottom flask was charged with 10 mL of anhydrous DMF and then 0.56 mL (11 mmol, 1.0 equiv) of bromine by syringe under a positive pressure of nitrogen. The reaction mixture was then cooled to −10° C. in an ice/methanol bath, and the bright red stock solution of oxidant was drawn into a degassed 12 mL disposable gas tight syringe and added to the substrate dropwise over 90 minutes with automation from a Ryzel syringe pump. At the end of the addition, the reaction mixture was diluted in succession with 50 mL of ice-cold deionized water and 75 mL of diethyl ether, the former of which resulted in bleaching of the solution's dark brown color. The resulting bilayer was transferred to a 125 mL separatory funnel, and after mixing, the yellow organic layer was removed. The aqueous layer, which was neutral to litmus paper, was washed two times with 30 mL portions of diethyl ether. In a 250 mL separatory funnel, the combined organic layers were washed three times with 100 mL portions of saturated aqueous sodium chloride in order to remove traces of DMF. The extract was dried over MgSO₄ and filtered through a medium porosity glass frit directly into a 250 mL round bottom flask in preparation for the thermal re-aromatization step. Concentration by rotary evaporation afforded a yellow solid that was immediately re-dissolved in toluene (55 mL), and the flask was equipped with a reflux condenser and a heating mantle. After 1 hour of stirring under air at reflux, the solvent was removed by rotary evaporation. Crude methyl olivetolate was purified by flash chromatography over silica gel with 5:1 petroleum ether: diethyl ether (R_{f}=0.35) as eluent and a KMnO₄ stain for spot visualization. Concentration of pure product fractions resulted in spontaneous crystallization of methyl olivetolate as a yellow solid (2.08 g, 80%) which was further dried under high vacuum. Mp-77-78° C.; IR (neat, cm⁻¹) 3600, 3400 (phenolic OH bands), 1660 (ester); ¹H NMR (CDCl₃, 500 MHz) δ 11.68 (s, 1H), 6.28 (d, J=2.9 Hz, 1H), 6.23 (d, J=2.5 Hz, 1H), 5.26 (br s, 1H), 3.92 (s, 3H), 2.83 (t, J=7.8 Hz, 2H), 1.56-1.49 (m, 2H), 1.36-1.30 (m, 4H), 0.90 (t, J=6.8 Hz, 3H);

¹³C NMR (CDCl₃, 125 MHz) δ 172.1, 165.4, 160.4, 149.1, 110.9, 104.8, 101.5, 52.1, 37.0, 32.2, 31.6, 22.7, 14.2. HRMS (ESI+) Calcd. for C₁₃H1904Na [M+H]⁺: 239.1283; Found 239.1280.

### Example 7: Synthesis of Enantiopure (−)-Cannabidiol (CBD) Via LAH Reduction

Enantiopure (−)-Cannabidiol (CBD) was prepared starting from commercially available (R)-limonene.

1. (S)-3-Methyl-6-(prop-1-en-2-yl)cyclohex-2-enone; common name (+)-(S)-isopiperitenone (Dethe, D. H. et al. 2015). A flame-dried, 100 mL round bottom flask was charged with 40 mL of tert-butanol by syringe. With vigorous magnetic stirring, 16.0 g (160 mmol, 4.0 equiv) of powdered chromium (VI) oxide was added in portions. Initial dissolution of the oxidant causes a bright orange solution to develop, but with continued stirring the solution becomes deep maroon in color and slight warming is observed. After 10 minutes at 25° C., the solution was cooled to 0° C., diluted with 40 mL of chloroform, and transferred to a 250 mL separatory funnel. Ice-cold DI water (50 mL) was added, and the mixture was agitated before allowing the phases to separate. The lower organic layer (bright red) was removed and the upper aqueous layer (light brown) was back-extracted twice with 20 mL portions of chloroform. The pooled organic layers were dried over sodium sulfate and vacuum filtered through cotton in a coarse porosity fritted funnel directly into a flame-dried 250 mL round bottom receiving flask. To the resulting deep red filtrate was added 6.50 mL (40.2 mmol, 1.0 equiv) of (R)-limonene. With continuous stirring, the vessel was equipped with a heating mantle and the solution brought to a gentle reflux. After 1 hour, the reaction mixture had become turbid and dark brown in color. The heat source was removed and stirring continued overnight at 25° C., at which point TLC analysis confirmed the absence of starting material and production of two regioisomeric enones. The mixture was directly filtered to remove insoluble Cr (IV) deposits, and the resulting brown filtrate was concentrated on a rotary evaporator in the presence of 10 grams of added silica gel. Purification of the residue by column chromatography with 10:1 petroleum ether: diethyl ether as eluent and a KMnO₄ stain for spot visualization furnished(S)-carvone as a viscous oil (R_{f}=0.55) as the major isomer (1.99 g, 33%) and isopiperitenone as a yellow liquid (R_{f}=0.40, 1.03 g, 17%). [α]²²D=+27.88 (c 0.54, CHCl₃); IR (neat, cm⁻¹) 3076, 2927, 1667, 1435, 1379, 1201, 1087, 891; 1H NMR (CDCl₃, 500 MHz) δ 5.89 (d, J=1.5 Hz, 1H), 4.94 (d, J=1.5 Hz, 1H), 4.75 (d, J=1.5 Hz, 1H), 2.94 (dd, J=10.5, 5.4 Hz, 1H), 2.35-2.31 (m, 2H), 2.11-1.98 (m, 2H), 1.94 (s, 3H), 1.74 (d, J=1.5 Hz, 3H); 13C NMR (CDCl₃, 125 MHZ) δ 199.5, 162.0; 143.4, 126.8, 113.6, 53.9, 30.4, 27.7, 24.3, 20.7; HRMS (ESI+) Calcd. for C₁₀H₁₄O [M]⁺: 150.1045; Found 150.1043.

2. (1R, 6S)-3-methyl-6-(prop-1-en-2-yl)cyclohex-2-enol, common name (±)-trans-isopiperitenol. A flame-dried, 100 mL round bottom flask was charged in air with 227 mg (5.98 mmol, 1.8 equiv) of lithium aluminum hydride (LAH) as a moisture-sensitive, gray powder. With magnetic stirring, 3.0 mL of anhydrous THF (dispensed from a solvent purification system) was added by syringe under a positive nitrogen pressure. The resulting heterogeneous suspension of reducing agent (2.0 M concentration) was cooled to −78° C. in a dry ice/acetone bath in dewar. A separate flame-dried, 50 mL pear bottom flask was charged with neat (+)-(S)-isopiperitenone (500 mg, 3.33 mmol, 1.0 equiv), and the ketone was dissolved in 10 mL of additional dry THF. The resulting pale yellow-orange solution of substrate was transferred to the LAH suspension down the walls of the receiving flask (for pre-cooling) with a 12 mL capacity degassed syringe. The rate of addition was controlled to offset modest bubbling observed in the reaction mixture, and the dewar bath temperature was kept at −78° C. by replenishment with additional dry ice. After 30 minutes of stirring post-addition, the reaction mixture was allowed to warm slowly to 0° C. and then held at that temperature for a quench by means of an ice bath. The reaction mixture was then treated in succession with 0.23 mL of deionized water, 0.23 mL of 10% (aq.) sodium hydroxide, and finally 0.69 ml of deionized water. The triplicate addition represents a convenient protocol for quenching n grams of LAH with n mL of H₂O, n mL of dilute NaOH, and 3n mL of H₂O to furnish granular precipitates of Al (OH) 3 salts that are easily removed by filtration, but it must be done with care due to an initially vigorous generation of hydrogen gas. After 30 minutes of continued stirring, the reaction mixture was filtered through a course fritted funnel and the precipitate and original vessel were washed two times with 5 mL of diethyl ether. The combined filtrate was concentrated to give a colorless liquid. ¹H NMR analysis of this mixture confirms the presence of 1,2-reduction products (±)-(1R, 6S)-isopiperitenol and (±)-(1S, 6S)-isopiperitenol in a ratio of 5:1 (trans: cis), with no detectable 1,4-reduction. Some separation of the major isomer is possible by flash chromatography in 5:1 petroleum ether:ethyl acetate with CAM staining for spot visualization. In the event, use of a 4 cm wide×16 cm tall silica gel column afforded 42.2 mg of (±)-trans-isopiperitenol (R_{f}=0.32) and 376 mg of a diastereomeric mixture (R_{f}=0.36, 83% total yield). Spectroscopic data for the minor (±)-cis-isopiperitenol was identical to material of 94% ee enriched in the (1R, 6R) enantiomer prepared previously by a multi-step asymmetric synthesis (Tomooka, K. et al. 2005): IR (neat, cm⁻¹) 3352, 3088, 2936, 1676, 1649, 1450, 1379, 1245, 1210, 1176, 1156, 1114, 1069, 1025, 957, 915, 886, 849, 816, 723, 681 cm⁻¹; ¹H NMR (CDCl₃, 500 MHZ) δ 5.67 (m, 1H), 4.99 (s, 1H), 4.81 (s, 1H), 4.12 (br s, 1H), 2.17-2.04 (m, 3H), 1.83 (s, 3H), 1.80-1.74 (m, 1H), 1.72 (s, 3H), 1.65-1.55 (m, 1H), 1.48 (br s, 1H); ¹³C NMR (CDCl₃, 125 MHZ) δ 146.7, 139.9, 122.5, 111.8, 63.9, 46.2, 31.3, 23.5, 22.7, 21.0. Characterization of the major diastereomer is as follows: IR (neat, cm⁻¹) 3396, 2925, 2854, 1453, 1375, 1035, 887; ¹H NMR (CDCl₃, 500 MHz) δ 5.45-5.44 (m, 1H), 4.89-4.88 (m, 1H), 4.85-4.84 (m, 1H), 4.13-4.10 (m, 1H), 2.10-2.04 (m, 2H), 1.95-1.93 (m, 1H), 1.73-1.72 (s, 3H), 1.69 (m, 3H), 1.65-1.56 (m, 2H); ¹³C NMR (CDCl₃, 125 MHz) δ 146.4, 136.6; 124.3, 112.2, 68.6, 50.8, 30.1, 26.1, 23.0, 19.3; HRMS (ESI+) Calcd. for C₁₀H₁₆O [M]⁺: 152.1201; Found 152.1209.

3. (−)-Cannabidiol acid methyl ester, common name methyl cannibidiolate. A flame-dried 1 dram vial was charged in air with 23.8 mg of methyl 2,4-dihydroxy-6-pentylbenzoate (0.100 mmol, 1.1 equiv) and 7.2 mg of camphorsulfonic acid (0.031 mmol, 0.34 equiv) as solids and then 0.6 mL of anhydrous dichloromethane was added as solvent after purging the vial with nitrogen gas. To the resulting clear solution stirring at 0° C. was added 13.8 mg (0.0907 mmol, 0.91 equiv) of (±)-trans-isopiperitenol in 0.4 mL of dichloromethane dropwise over 1 hour. The reaction mixture was stirred for an additional hour at 0° C., the ice bath removed, and a nitrogen gas purge line (and vent needle) was used to concentrate the reaction mixture to a neat oil at 25° C. The vial contents were then resuspended in 1.0 mL of diethyl ether, 1.0 mL of cold saturated (aq.) sodium bicarbonate was added to quench the acid catalyst, and after agitation by stirring the organic layer was drawn out by Pasteur pipette. The aqueous layer was washed 2× with 1.0 mL of diethyl ether and the extracts pooled, dried over MgSO₄, filtered through cotton, and concentrated to an oily residue. Purification by silica gel chromatography in 9:1 petroleum ether: diethyl ether furnished the title compound as a yellow oil (21.1 mg, 60%, R_{f}=0.55, UV active spot or dark blue to CAM stain). ¹H NMR (CDCl₃, 500 MHZ) δ 12.00 (s, 1H), 6.51 (s, 1H), 6.21 (s, 1H), 5.55 (s, 1H), 4.52 (s, 1H), 4.37 (s, 1H), 4.10 (br s, 1H), 3.90 (s, 3H), 2.87-2.81 (m, 1H), 2.76-2.69 (m, 1H), 2.71-2.65 (m, 1H), 2.42-2.32 (m, 1H), 2.34-2.27 (m, 1H), 2.26-2.17 (m, 1H), 2.12-2.06 (m, 1H), 1.79 (s, 3H), 1.71 (s, 3H), 1.54-1.46 (m, 2H), 1.35-1.27 (m, 2H), 1.27-1.23 (m, 2H), 0.89 (t, J=6.5 Hz, 3H). If the above protocol is carried out with the >4:1 trans: cis mixture of (+)-isopiperitenols, the minor diastereomer is consumed and similar yield is obtained.

4. (−)-Cannabidiol (CBD). The methyl cannibidiolate from above (21.1 mg, 0.0566 mmol, 1.0 equiv) was dissolved in 0.28 mL of methanol in a 1 dram vial containing a teflon-coated flea stir bar. With magnetic stirring, the solution was treated with 0.22 mL of a 0.5 N standardized sodium hydroxide solution by syringe through a rubber septum under positive nitrogen pressure. The septum was removed, the opening sealed with a Teflon-lined screw-cap closure, and the vial submerged in an oil bath heated to 65° C. The reaction mixture was stirred continuously for three hours under gentle reflux and then cooled to 25° C., at which point a TLC analysis confirmed the absence of starting ester. The mixture was acidified by adding 1.0 mL of a 30% solution of citric acid in deionized water, and the contents were washed 3× with 0.5 mL volumes of diethyl ether. Liquid-liquid extraction was conveniently performed in the same vial by capping and shaking the two phases. The siphoned and combined organic washes were dried over MgSO₄, filtered through cotton, and concentrated to provide the title compound CBD as an off-white solid (16 mg, 90%). [α]²²D=−122 (c 1.0, EtOH); IR (neat, cm⁻¹) 3513, 3401, 2914, 1621, 1438, 1216; ¹H NMR (CDCl₃, 500 MHz) δ 6.40-6.10 (br s, 2H), 6.10-5.80 (br s, 1H), 5.57 (s, 1H), 4.90-4.60 (br s, 1H), 4.64 (m, 1H), 4.54 (m, 1H), 3.90-3.80 (dm, J=11.8 Hz, 1H), 2.50-2.40 (m, 3H), 2.30-2.00 (m, 2H), 1.90-1.75 (m, 2H), 1.82 (s, 3H), 1.67 (s, 3H), 1.65-1.50 (m, 2H), 1.40-1.20 (m, 4H), 0.90 (t, J=6.5 Hz, 3H); ¹³C NMR (DMSO-d₆, 125 MHz) δ 156.7, 149.6; 140.6, 130.5, 127.3, 114.6, 110.1, 107.1, 44.1, 36.0, 35.4, 31.5, 30.8, 30.0, 23.8, 22.5, 19.7, 14.4; HRMS (ESI+) Calcd. for C₂₁H₂₉O₂ [M−H]⁻: 313.2173; Found 313.2170.

### Example 8: Synthesis of Racemic (±)-Cannabidiol (CBD) Via (±)-Trans-Isopiperitenol

The methods described in Example 1 were adapted to provide racemic (±)-Cannabidiol (CBD). In particular, the following method was used to provide (±)-trans-isopiperitenol in >5:1 dr, which is then subjected to Steps 3 and 4 of Example 7.

1. Rac-(±)-3-Methyl-6-(prop-1-en-2-yl)cyclohex-2-enone, common name (=)-isopiperitenone. A 1.75 gram sample of commercial citral (≥96%, mixture of trans (geranial) and cis (neral), was filtered through a column (5 cm diameter×10 cm high) of silica gel slurry-packed in benzene containing 3% diethyl ether (R_{f}=0.54; UV active or yellow spot under KMnO₄ staining). This flash chromatography step removes a yellow-colored impurity that elutes with the solvent front, and it also serves to rigorously dry the substrate due to azeotropic removal of any traces of water. Concentration of pure citral fractions on a rotary evaporator, backfilling of the apparatus with an argon balloon, and further degassing under high vacuum and with magnetic stirring of the neat oil furnished 1.52 g (10.0 mmol, 1.0 equiv) of citral that was completely colorless. The starting material was dissolved in 10.0 mL of anhydrous dichloromethane, and a 12 mL-capacity plastic syringe was charged with the solution. With degassing under positive nitrogen pressure, a second disposable gas-tight syringe was loaded with commercial dimethylaluminum chloride (10.1 mL of 1.0 M in hexanes, 10.1 mmol, 1.01 equiv). Lastly, a separate flame-dried, 24/40 jointed, 100 mL round bottom flask with a side-arm stopcock and hose connector (for nitrogen pressure) was charged with 10.0 mL of dichloromethane and cooled to −10° C. in an ice/methanol bath. At the same addition rates using two syringe pumps, the substrate and promoter solutions were added dropwise to the chilled flask over three hours. The exit needle for the promoter solution was placed below the surface of solvent to minimize smoking or cloudiness in the headspace of the reaction flask. The role of slow addition is to mimic high dilution and minimize intermolecular reactions. Over the course of the addition, the reaction mixture turns light orange and eventually maroon in color. Stirring was continued with slow warming to room temperature for one hour. The mixture was then quenched at 0° C. by the sequential addition of 1.0 mL of DI water, 1.0 mL of 4 M aqueous sodium hydroxide, and an additional 3.0 mL of water. These additions, which mirror a standard workup for a lithium aluminum hydride reaction, cause methane gas evolution, bleaching of the orange color in the reaction mixture, and formation of a milky precipitate from which the yellow supernatant was easily isolated by decantation. Thus, filtration of the mixture through a pad of cotton, washing of the Al (III) hydroxide precipitate with extra dichloromethane, and concentration provided a bronze oil. ¹H NMR analysis of the crude reaction mixture showed 30% conversion of citral to a product mixture consisting of a 2.5:1 ratio of (±)-isopiperitenone to (±)-isopiperitenols, with the latter present in >10:1 dr on the basis of the predicted trans-selective carbonyl-ene reaction promoted by the Al reagent. However, mechanistic considerations suggest that following the designed pericyclic process, the electron-rich dimethylaluminum (III) alkoxide undergoes an internal Oppenauer oxidation (via Meerwein-Ponndorf-Verley hydride transfer to unreacted citral), leading to ketone as the major product. The desired polar fraction (R_{f}=0.38 in 2:1 heptane: diethyl ether) was separated from unreacted citral by flash filtration through silica gel in the same eluent with either KMnO₄ or ceric ammonium molybdate (CAM) TLC staining. This allowed >60% mass recovery of citral (R_{f}=0.90) for recycling and 228 mg (15% yield) of the 2.5:1 (±)-isopiperitenone: (±)-isopiperitenols mix as a colorless oil. Subjection of this mixture to the 1,2-reduction protocol exactly as reported above (Example 7, step 1) allowed interception of the asymmetric route to CBD. Spectroscopic and TLC analytical data for (±)-isopiperitenone, (±)-trans-isopiperitenol, and (±)-cis-isopiperitenol were identical to those of the optically active standards (+)-(S)-isopiperitenone, (±)-(1R, 6S)-isopiperitenol, and (±)-(15, 6S)-isopiperitenol as reported above in steps 1 and 2 of Example 7.

### Example 9: Synthesis of Enantiopure (−)-Delta-9-Tetrahydrocannabinol (Δ⁹-THC) Via LAH Reduction

Enantiopure (−)-delta-9-Tetrahydrocannabinol (Δ⁹-THC) was prepared starting from commercially available (R)-limonene.

1. (S)-3-Methyl-6-(prop-1-en-2-yl)cyclohex-2-enone; common name (+)-(S)-isopiperitenone (Dethe, D. H. et al. 2015). A flame-dried, 100 mL round bottom flask was charged with 40 mL of tert-butanol by syringe. With vigorous magnetic stirring, 16.0 g (160 mmol, 4.0 equiv) of powdered chromium (VI) oxide was added in portions. Initial dissolution of the oxidant causes a bright orange solution to develop, but with continued stirring the solution becomes deep maroon in color and slight warming is observed. After 10 minutes at 25° C., the solution was cooled to 0° C., diluted with 40 mL of chloroform, and transferred to a 250 mL separatory funnel. Ice-cold DI water (50 mL) was added, and the mixture was agitated before allowing the phases to separate. The lower organic layer (bright red) was removed and the upper aqueous layer (light brown) was back-extracted twice with 20 mL portions of chloroform. The pooled organic layers were dried over sodium sulfate and vacuum filtered through cotton in a coarse porosity fritted funnel directly into a flame-dried 250 mL round bottom receiving flask. To the resulting deep red filtrate was added 6.50 mL (40.2 mmol, 1.0 equiv) of (R)-limonene. With continuous stirring, the vessel was equipped with a heating mantle and the solution brought to a gentle reflux. After 1 hour, the reaction mixture had become turbid and dark brown in color. The heat source was removed and stirring continued overnight at 25° C., at which point TLC analysis confirmed the absence of starting material and production of two regioisomeric enones. The mixture was directly filtered to remove insoluble Cr (IV) deposits, and the resulting brown filtrate was concentrated on a rotary evaporator in the presence of 10 grams of added silica gel. Purification of the residue by column chromatography with 10:1 petroleum ether: diethyl ether as eluent and a KMnO₄ stain for spot visualization furnished(S)-carvone as a viscous oil (R_{f}=0.55) as the major isomer (1.99 g, 33%) and isopiperitenone as a yellow liquid (R_{f}=0.40, 1.03 g, 17%). [α]²²D=+27.88 (c 0.54, CHCl₃); IR (neat, cm⁻¹) 3076, 2927, 1667, 1435, 1379, 1201, 1087, 891; ¹H NMR (CDCl₃, 500 MHZ) 8 5.89 (d, J=1.5 Hz, 1H), 4.94 (d, J=1.5 Hz, 1H), 4.75 (d, J=1.5 Hz, 1H), 2.94 (dd, J=10.5, 5.4 Hz, 1H), 2.35-2.31 (m, 2H), 2.11-1.98 (m, 2H), 1.94 (s, 3H), 1.74 (d, J=1.5 Hz, 3H); 13C NMR (CDCl₃, 125 MHZ) δ 199.5, 162.0; 143.4, 126.8, 113.6, 53.9, 30.4, 27.7, 24.3, 20.7; HRMS (ESI+) Calcd. for C₁₀H₁₄O [M]⁺: 150.1045; Found 150.1043.

2. (1R, 6S)-3-methyl-6-(prop-1-en-2-yl)cyclohex-2-enol, common name (±)-trans-isopiperitenol. A flame-dried, 100 mL round bottom flask was charged in air with 227 mg (5.98 mmol, 1.8 equiv) of lithium aluminum hydride (LAH) as a moisture-sensitive, gray powder. With magnetic stirring, 3.0 mL of anhydrous THF (dispensed from a solvent purification system) was added by syringe under a positive nitrogen pressure. The resulting heterogeneous suspension of reducing agent (2.0 M concentration) was cooled to −78° C. in a dry ice/acetone bath in dewar. A separate flame-dried, 50 mL pear bottom flask was charged with neat (+)-(S)-isopiperitenone (500 mg, 3.33 mmol, 1.0 equiv), and the ketone was dissolved in 10 mL of additional dry THF. The resulting pale yellow-orange solution of substrate was transferred to the LAH suspension down the walls of the receiving flask (for pre-cooling) with a 12 mL capacity degassed syringe. The rate of addition was controlled to offset modest bubbling observed in the reaction mixture, and the dewar bath temperature was kept at −78° C. by replenishment with additional dry ice. After 30 minutes of stirring post-addition, the reaction mixture was allowed to warm slowly to 0° C. and then held at that temperature for a quench by means of an ice bath. The reaction mixture was then treated in succession with 0.23 mL of deionized water, 0.23 mL of 10% (aq.) sodium hydroxide, and finally 0.69 ml of deionized water. The triplicate addition represents a convenient protocol for quenching n grams of LAH with n mL of H₂O, n mL of dilute NaOH, and 3n mL of H₂O to furnish granular precipitates of Al(OH)₃ salts that are easily removed by filtration, but it must be done with care due to an initially vigorous generation of hydrogen gas. After 30 minutes of continued stirring, the reaction mixture was filtered through a course fritted funnel and the precipitate and original vessel were washed two times with 5 mL of diethyl ether. The combined filtrate was concentrated to give a colorless liquid. ¹H NMR analysis of this mixture confirms the presence of 1,2-reduction products (±)-(1R, 6S)-isopiperitenol and (±)-(15, 6S)-isopiperitenol in a ratio of 5:1 (trans: cis), with no detectable 1,4-reduction. Some separation of the major isomer is possible by flash chromatography in 5:1 petroleum ether:ethyl acetate with CAM staining for spot visualization. In the event, use of a 4 cm wide×16 cm tall silica gel column afforded 42.2 mg of (±)-trans-isopiperitenol (R_{f}=0.32) and 376 mg of a diastereomeric mixture (R=0.36, 83% total yield). Spectroscopic data for the minor (±)-cis-isopiperitenol was identical to material of 94% ee enriched in the (1R, 6R) enantiomer prepared previously by a multi-step asymmetric synthesis (Tomooka, K. et al. 2005): IR (neat, cm⁻¹) 3352, 3088, 2936, 1676, 1649, 1450, 1379, 1245, 1210, 1176, 1156, 1114, 1069, 1025, 957, 915, 886, 849, 816, 723, 681 cm⁻¹; ¹H NMR (CDCl₃, 500 MHZ) δ 5.67 (m, 1H), 4.99 (s, 1H), 4.81 (s, 1H), 4.12 (br s, 1H), 2.17-2.04 (m, 3H), 1.83 (s, 3H), 1.80-1.74 (m, 1H), 1.72 (s, 3H), 1.65-1.55 (m, 1H), 1.48 (br s, 1H); ¹³C NMR (CDCl₃, 125 MHZ) δ 146.7, 139.9, 122.5, 111.8, 63.9, 46.2, 31.3, 23.5, 22.7, 21.0. Characterization of the major diastereomer is as follows: IR (neat, cm⁻¹) 3396, 2925, 2854, 1453, 1375, 1035, 887; ¹H NMR (CDCl₃, 500 MHz) δ 5.45-5.44 (m, 1H), 4.89-4.88 (m, 1H), 4.85-4.84 (m, 1H), 4.13-4.10 (m, 1H), 2.10-2.04 (m, 2H), 1.95-1.93 (m, 1H), 1.73-1.72 (s, 3H), 1.69 (m, 3H), 1.65-1.56 (m, 2H); ¹³C NMR (CDCl₃, 125 MHZ) δ 146.4, 136.6; 124.3, 112.2, 68.6, 50.8, 30.1, 26.1, 23.0, 19.3; HRMS (ESI+) Calcd. for C₁₀H₁₆O [M]⁺: 152.1201; Found 152.1209.

3. (−)-delta-9-Tetrahydrocannabinol (Δ⁹-THC), international nonproprietary name Dronabinol. para-Toluenesulfonic acid monohydrate was recrystallized (as thin needles) from boiling chloroform, washed with hexane, and dried in vacuo prior to usage. A flame-dried 10 mL round bottom flask was charged with 20.0 mg of commercial olivetol (0.111 mmol, 1.0 equiv) and 6.8 mg of p-TsOH (0.039 mmol, 0.35 equiv) as solids and 1.7 mL of anhydrous dichloromethane under positive pressure of nitrogen. To the resulting clear solution stirring at 25° C. was added 18.6 mg (0.122 mmol, 1.1 equiv) of (±)-trans-isopiperitenol (Cardillo, B. et al. 1973) in 0.5 mL of dichloromethane dropwise over 1 hour. TLC analysis of the mixture after 2 hours of reaction showed only trace amounts of the reactants and a prominent non-polar spot (R_{f}=>0.90 in 3:1 hexane: diethyl ether). The reaction was quenched with 2.0 mL of saturated sodium bicarbonate and the organic layer removed. The water layer was washed 2x with 1.0 mL dichloromethane and the extracts combined, dried over MgSO₄, filtered through cotton, and concentrated to an oily residue. Purification by passage through silica gel in 50:1 petroleum ether:ethyl acetate delivered 12.7 mg (33%) of a clean 3:1 mixture of Δ⁹-THC and Δ⁸-THC (Hively, R. L. et al. 1966) as the first compounds to elute. Separation by HPLC has been reported under the following conditions: Sunfire C18 5μ, 4.6×150 mm column, 1 mL/min flow rate, gradient elution 80% water in acetonitrile to 100% acetonitrile containing 0.5-1% TFA. Spectroscopic and analytical data for synthetic Δ⁹-THC match that of both the natural product (Gaoni, Y. et al. 1964) and racemic material prepared according to an early biomimetic total synthesis (Taylor, E. C. et al. 1966).

### Example 10: Synthesis of Racemic (±)-Delta-9-Tetrahydrocannabinol (Δ⁹-THC) Via (±)-Trans-Isopiperitenol

The methods described in Example 9 were adapted to provide racemic (±)-delta-9-Tetrahydrocannabidiol (Δ⁹-THC). In particular, the following method was used to provide (±)-trans-isopiperitenol in >5:1 dr, which is then subjected to Step 3 of Example 9.

1. Rac-(±)-3-Methyl-6-(prop-1-en-2-yl)cyclohex-2-enone, common name (=)-isopiperitenone. A 1.75 gram sample of commercial citral (>96%, mixture of trans (geranial) and cis (neral), was filtered through a column (5 cm diameter×10 cm high) of silica gel slurry-packed in benzene containing 3% diethyl ether (R_{f}=0.54; UV active or yellow spot under KMnO₄ staining). This flash chromatography step removes a yellow-colored impurity that elutes with the solvent front, and it also serves to rigorously dry the substrate due to azeotropic removal of any traces of water. Concentration of pure citral fractions on a rotary evaporator, backfilling of the apparatus with an argon balloon, and further degassing under high vacuum and with magnetic stirring of the neat oil furnished 1.52 g (10.0 mmol, 1.0 equiv) of citral that was completely colorless. The starting material was dissolved in 10.0 mL of anhydrous dichloromethane, and a 12 mL-capacity plastic syringe was charged with the solution. With degassing under positive nitrogen pressure, a second disposable gas-tight syringe was loaded with commercial dimethylaluminum chloride (10.1 mL of 1.0 M in hexanes, 10.1 mmol, 1.01 equiv). Lastly, a separate flame-dried, 24/40 jointed, 100 mL round bottom flask with a side-arm stopcock and hose connector (for nitrogen pressure) was charged with 10.0 mL of dichloromethane and cooled to −10° C. in an ice/methanol bath. At the same addition rates using two syringe pumps, the substrate and promoter solutions were added dropwise to the chilled flask over three hours. The exit needle for the promoter solution was placed below the surface of solvent to minimize smoking or cloudiness in the headspace of the reaction flask. The role of slow addition is to mimic high dilution and minimize intermolecular reactions. Over the course of the addition, the reaction mixture turns light orange and eventually maroon in color. Stirring was continued with slow warming to room temperature for one hour. The mixture was then quenched at 0° C. by the sequential addition of 1.0 mL of DI water, 1.0 mL of 4 M aqueous sodium hydroxide, and an additional 3.0 mL of water. These additions, which mirror a standard workup for a lithium aluminum hydride reaction, cause methane gas evolution, bleaching of the orange color in the reaction mixture, and formation of a milky precipitate from which the yellow supernatant was easily isolated by decantation. Thus, filtration of the mixture through a pad of cotton, washing of the Al (III) hydroxide precipitate with extra dichloromethane, and concentration provided a bronze oil. ¹H NMR analysis of the crude reaction mixture showed 30% conversion of citral to a product mixture consisting of a 2.5:1 ratio of (±)-isopiperitenone to (±)-isopiperitenols, with the latter present in >10:1 dr on the basis of the predicted trans-selective carbonyl-ene reaction promoted by the Al reagent. However, mechanistic considerations suggest that following the designed pericyclic process, the electron-rich dimethylaluminum (III) alkoxide undergoes an internal Oppenauer oxidation (via Meerwein-Ponndorf-Verley hydride transfer to unreacted citral), leading to ketone as the major product. The desired polar fraction (R_{f}=0.38 in 2:1 heptane: diethyl ether) was separated from unreacted citral by flash filtration through silica gel in the same eluent with either KMnO₄ or ceric ammonium molybdate (CAM) TLC staining. This allowed >60% mass recovery of citral (R_{f}=0.90) for recycling and 228 mg (15% yield) of the 2.5:1 (±)-isopiperitenone: (±)-isopiperitenols mix as a colorless oil. Subjection of this mixture to the 1,2-reduction protocol exactly as reported above (Example 1, step 1) allowed interception of the asymmetric route to CBD. Spectroscopic and TLC analytical data for (±)-isopiperitenone, (±)-trans-isopiperitenol, and (±)-cis-isopiperitenol were identical to those of the optically active standards (+)-(S)-isopiperitenone, (±)-(1R, 6S)-isopiperitenol, and (±)-(15, 6S)-isopiperitenol as reported above in steps 1 and 2 of Example 7.

### DISCUSSION

Cannabidiol is well-known phytocannabinoid produced as a primary constituent in both marijuana and hemp plants Cannabis sativa and Cannabis indica. Over several decades, the endocannabinoid system of receptors (ECS) underlying the physiological effects of Cannabis botanicals in humans has emerged as a premier target of pharmacotherapy (Mechoulam, R. et al. 2014). The ECS proteins include cannabinoid receptors CB₁ and CB₂ as well as endogenous ligands named endocannabinoids (Morales, P. et al. 2017). The CB₁ receptor is abundant in the brain but to a lower extent in peripheral tissues, whereas CB₂ is expressed primarily on the surface of circulating immune cells. Presently, cannabidiol (CBD) is generating a surge of medical interest because of its demonstrated antiepileptic, anxiolytic, antipsychotic, anti-inflammatory, and neuroprotective properties. Manufacture of CBD on a commercial scale presents a number of challenges, however, due to the universal preparative approach that relies on Friedel-Crafts alkylation of olivetol (1,3-dihydroxy-5-pentylbenzene) to install the key aryl-monoterpenyl C—C bond. One critical problem plaguing a number of syntheses is the requirement for strong Lewis or Bronsted acidic reagents to promote terpenylation; acidic conditions make it impossible to stop further cyclization of the phenol group into the dihydropyran ring of (−)-trans-delta-9-tetrahydrocannabinol (THC), a Schedule 1, federally controlled substance. Additional isomers such as cis-Δ⁹-THC (a dihydropyran stereoisomer), Δ⁸-THC (deriving from migration of the cyclohexene double bond), and iso-THC (a regioisomeric material resulting from F—C alkylation ortho to the 5C n-amyl substituent) are tedious to separate from the desired product, adding time and costs and making it difficult to achieve current standards of purity for active pharmaceutical ingredients. Extraction and purification of naturally occurring CBD is economically less attractive and presents similar drawbacks. For instance, even industrial strains of hemp bred specifically to harbor low quantities of psychoactive Δ⁹-THC often exceed the legal limit of ≤ 0.3% by dry mass.

It is compelling that in spite of its notorious status as a federally regulated drug with a high potential for abuse, trans-Δ⁹-THC, which goes by the generic name dronabinol, has widespread medical utility as an antiemetic agent (appetite stimulant) and a sleep apnea reliever. Clinical usage is also FDA-approved for the treatment of HIV/AIDS-induced anorexia, chemotherapy-induced nausea, and glaucoma. An additional feature that speaks further to regulatory and agricultural issues in the Cannabis industry stems from the fact that both Δ⁹-THC and CBD are biosynthesized in the plants along completely parallel yet independent pathways from the corresponding carboxylic acids (Δ⁹-THC acid and cannabidiolic acid, CBDA). The exact environmental (crop harvesting) and physiological conditions that permit spontaneous decarboxylation (loss of carbon dioxide) from each endogenous precursor are not fully understood. At the same time, these variables are acknowledged as likely contributors to differences in biological effects as a function of how the therapeutic agents are formulated and ingested. For example, the mature flowers of female Cannabis plants concentrate varying amounts of both Δ⁹-THC acid and CBDA, but how quickly these molecules convert to THC and CBD is dependent upon sunlight exposure and time left standing at ambient temperatures. If buds of the plant are ingested, a human patient experiences a unique duration and intensity of pharmacological effects. By contrast, smoking or vaporizing the plant material results in instantaneous decarboxylation due to the higher temperature, and the patient's experience stems from inhalation and a faster absorption of THC and CBD into the bloodstream in the lungs.

The present invention is directed towards overcoming a number of inefficiencies related not only to the cost, availability, and composition of these pharmaceutical agents, but also with regards to standardized or controlled modes of administration to patients seeking diverse medicinal benefits. Specifically, a preferred embodiment of the invention provides a novel process for synthesizing CBD in a stereo- and regiochemically controlled manner with CBD acid as a direct synthetic precursor-just as it is in Nature. Along with CBD, CBDA can be tested and advanced as a valuable pro-drug, one expected to have better water solubility (relative to CBD) for formulating topical creams and lotions in health and beauty products as well as for dietary supplements, food, and beverages. Additionally, processes reported herein establish clean and reliable transformation of synthetic CBDA into CBD on scales and levels of purity that promote safer, uniform supplies of this increasingly popular nutriceutical.

### REFERENCES

Aguillón, A. R., Leão, R. A. C., Miranda, L. S. M., de Souza, R. O. M. A. Cannabidiol Discovery and Synthesis—A Target-Oriented Analysis in Drug Production Processes. Chem. Eur. J. 2021, 27, 5577-5600.
Baek, S-H., Srebnik, M., Mechoulam, R. Tetrahedron Lett. 1985, 26, 1083-1086.
Burdick, D. C., Collier, S. J., Biolatto, B., Mecklar, H. Process for Production of Delta-9-Tetrahydrocannibinol. U.S. Pat. No. 8,106,244 B2, Jan. 31, 2012.
Cardillo, B., Merlini, L., Servi, S. Alkylation of Resorcinols with Monoterpenoid Allylic Alcohols in Aqueous Acid. Synthesis of New Cannabinoid Derivatives. Gazz. Chim. Ital. 1973, 103, 127. To our knowledge, this citation has the earliest example of olivetol terpenylation with p-mentha-1,8-dien-3-ol, and it was effected with aqueous acid. Preparation of the monoterpenoid starting material involved a number of steps. A cis/trans mixture of isopiperitenol has also been converted to Δ⁹-THC with boron trifluoride diethyl etherate and olivetol, but in our hands Lewis acid catalysis leads to competitive amounts of the Friedel-Crafts positional isomer iso-Δ⁹-THC.
Chan, T-H., Brownbridge, P. A Novel Cycloaromatization Reaction. Regiocontrolled Synthesis of Substituted Methyl Salicylates. J. Am. Chem. Soc. 1980, 102, 3534-3538.
Dethe, D. H., Erande, R. D., Mahapatra, S., Das, S., Kumar, K. B. Protecting Group Free Enantiospecific Total Syntheses of Structurally Diverse Natural Products of the Tetrahydrocannabinoid Family. Chem. Comm. 2015, 51, 2871-2873.
Focella, A., Teitel, S., Brossi, A. A Simple and Practical Synthesis of Olivetol. J. Org. Chem. 1977, 42, 3456-3457.
Gaoni, Y., Mechoulam, R. Isolation, Structure, and Partial Synthesis of an Active Constituent of Hashish. J. Am. Chem. Soc. 1964, 86, 1646-1647.
Hanus, L. O., Meyer, S. M., Muñoz, E., Taglialatela-Scafati, O., Appendino, G. Phytocannabinoids: A Critical Inventory. Nat. Prod. Rep. 2016, 33, 1357-1392.
Hively, R. L., Mosher, W. A., Hoffmann, F. W. Isolation of A6-Tetrahydrocannabinol from Marijuana. J. Am. Chem. Soc. 1966, 88, 1832-1833. Named Δ⁶-3,4-trans-THC under an alternative numbering scheme, double bond isomerization to the thermodynamic Δ⁸-position is precedented in acidic conditions, and it can also occur through physiology or in isolation work.
Mechoulam, R., Gaoni, Y. Hashish. IV. The Isolation and Structure of Cannabinolic, Cannabidiolic, and Cannabigerolic Acids. Tetrahedron 1965, 21, 1223-1229.
Mechoulam, R.; Hanus, L. O.; Pertwee, R.; Howlett, A. C. “Early Phytocannabinoid Chemistry to Endocannabinoids and Beyond,” Nat. Rev. Neurosci. 2014, 15, 757-764.
Morales, P.; Reggio, P. H.; Jagerovic, N. “An Overview on Medicinal Chemistry of Synthetic and Natural Derivatives of Cannabidiol,” Front. Pharmacol. 2017, 8, 422-450.
Petrzilka, T., Haefliger, W., Sikemeier, G., Ohloff, G., Eschenmoser, A. Synthese und Chiralität des(−)-Cannabidiols Vorläufige Mitteilung. Helv. Chim. Acta 1967, 50, 719-723.
Souza, F. E. S., Field, J. E., Pan, M., Ramjit, N. J., Tharmanathan, T., Jende-Tindall, T. Synthetic Route to Dronabinol. U.S. Pat. No. 7,323,576 B2, Jan. 29, 2008.
Taylor, E. C., Lenard, K., Shvo, Y. Active Constituents of Hashish. Synthesis of dl-Δ⁶-3,4-trans-Tetrahydrocannabinol. J. Am. Chem. Soc. 1966, 88, 367-369.
Tomooka, K., Komine, N., Fujiki, D., Nakai, T., Yanagitsuru, S-I. Planar Chiral Cyclic Ether: Asymmetric Resolution and Chirality Transformation. J. Am. Chem. Soc. 2005, 127, 12182-12183.
Womack, E. B., Nelson, A. B. Ethyl Diazoacetate. Org. Synth. 1944, 24, 56.

What is claimed is:

1. A process for producing a compound having the structure of formula (−)-(I): 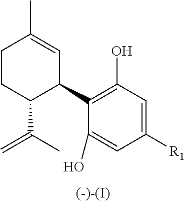
wherein
R₁ is alkyl,
the process comprising:
(i) reacting (R)-limonene with an oxidizing agent in a first suitable solvent under conditions sufficient to produce a compound having the structure of formula (+)-(IIa): 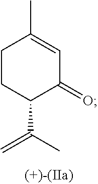
and
(ii) reacting the compound having the structure of formula (+)-(IIa) produced in step (i) with a reducing agent in a second suitable solvent under conditions sufficient to produce a compound having the structure of formula (+)-(II′) as a single enantiomer or (+)-(II″) as a single enantiomer:

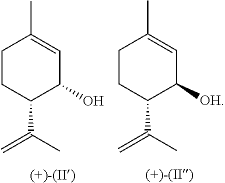

2. The process of claim 1, wherein the oxidizing agent comprises a chromium metal complex or salt.

3. The process of claim 2, wherein the oxidizing agent is chromium (VI) oxide.

4. The process of any one of claims 1-3, wherein the first suitable solvent is chloroform.

5. The process of any one of claims 1-4, wherein the reducing agent comprises sodium metal.

6. The process of claim 5, wherein the reducing agent is sodium borohydride.

7. The process of claim 5 or 6, wherein the second suitable solvent is methanol.

8. The process of any one of claims 5-7, wherein the reaction of step (ii) occurs in the presence of cerium (III) chloride or samarium (III) iodide.

9. The process of any one of claims 1-8, wherein the reducing agent comprises lithium metal.

10. The process of claim 9, wherein the reducing agent is lithium aluminum hydride.

11. The process of claim 9 or 10, wherein the second suitable solvent is tetrahydrofuran.

12. The process of any one of claims 9-11, wherein the reaction of step (ii) occurs at a temperature of less than 0° C.

13. The process of any one of claims 1-12, further comprising:
(iii) reacting the compound having the structure of formula (+)-(II′) or (+)-(II″) produced in step (ii) with a compound having the structure of formula (III) in the presence of an acid in a suitable solvent: 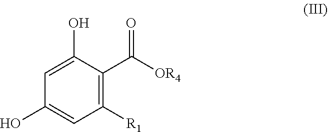
wherein R₄ is alkyl, under conditions sufficient to produce a compound having the structure of formula (−)-(IV):

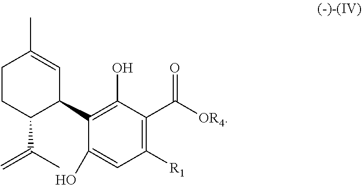

14. The process of claim 13, wherein the acid is an organic acid or Lewis acid.

15. The process of claim 14, wherein the acid is camphorsulfonic acid.

16. The process of any one of claims 13-15, wherein the suitable solvent is dichloromethane.

17. The process of any one of claims 13-16, further comprising:
(iv) subjecting the compound having the structure of formula (−)-(IV) produced in step (iii) to basic hydrolysis conditions sufficient to produce a compound having the structure of formula (−)-(V): 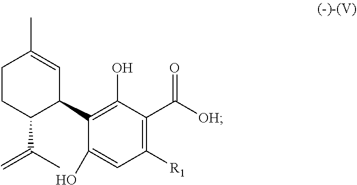
and
(v) subjecting the compound having the structure of formula (−)-(V) produced in step (iv) to decarboxylation conditions sufficient to produce the compound having the structure of formula (−)-(I).

18. The process of any one of claims 13-17, wherein the compound having the structure of formula (III) is prepared by a process comprising:
(a) reacting a compound having the structure of formula (VIa): 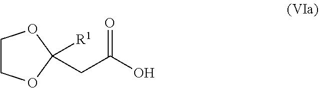
with an activating agent in a first suitable solvent under conditions sufficient to produce a compound having the structure of formula (VIb): 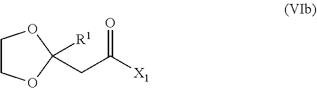
wherein
X₁ is a halide or activating ester moiety;
(b) reacting the compound having the structure of formula (VIb) with a diene having the structure of formula (VIc) in the presence of a Lewis acid in a second suitable solvent: 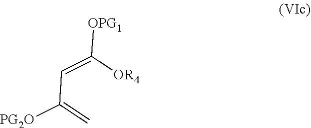
wherein
PG₁ and PG₂ are each independently a hydroxyl protecting group; and
R₄ is alkyl, under conditions sufficient to form the cyclized compound having the structure of formula (III).

19. The process of any one of claims 1-18, wherein R₁ is n-pentyl.

20. A process for producing a compound having the structure of formula (−)-(Ia): 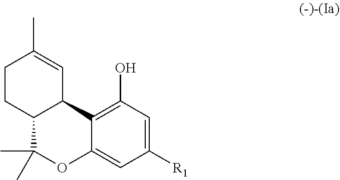
wherein
R₁ is alkyl,
the process comprising:
(i) reacting (R)-limonene with an oxidizing agent in a first suitable solvent under conditions sufficient to produce a compound having the structure of formula (+)-(IIa): 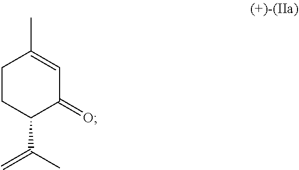
and
(ii) reacting the compound having the structure of formula (+)-(IIa) produced in step (i) with a reducing agent in a second suitable solvent under conditions sufficient to produce a compound having the structure of formula (+)-(II′) as a single enantiomer or (+)-(II″) as a single enantiomer:

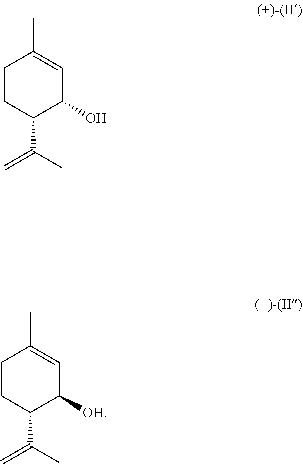

21. The process of claim 20, wherein the oxidizing agent comprises a chromium metal complex or salt.

22. The process of claim 21, wherein the oxidizing agent is chromium (VI) oxide.

23. The process of any one of claims 20-22, wherein the first suitable solvent is chloroform.

24. The process of any one of claims 20-23, wherein the reducing agent comprises sodium metal.

25. The process of claim 24, wherein the reducing agent is sodium borohydride.

26. The process of claim 24 or 25, wherein the second suitable solvent is methanol.

27. The process of any one of claims 24-26, wherein the reaction of step (ii) occurs in the presence of cerium (III) chloride or samarium (III) iodide.

28. The process of any one of claims 20-23, wherein the reducing agent comprises lithium metal.

29. The process of claim 28, wherein the reducing agent is lithium aluminum hydride.

30. The process of claim 28 or 29, wherein the second suitable solvent is tetrahydrofuran.

31. The process of any one of claims 28-30, wherein the reaction of step (ii) occurs at a temperature of less than 0° C.

32. The process of any one of claims 20-31, further comprising:
(iii) reacting the compound having the structure of formula (+)-(II′) or (+)-(II″) produced in step (ii) with a compound having the structure of formula (IIIa) in the presence of an organic acid or a Lewis acid in a second suitable solvent: 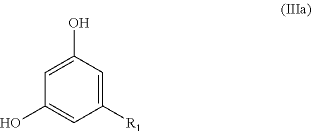
so as to thereby produce the compound having the structure of formula (−)-(Ia).

33. The process of claim 32, wherein the acid is an organic acid or a Lewis acid.

34. The process of claim 33, wherein the acid is p-toluenesulfonic acid.

35. The process of any one of claims 32-34, wherein the suitable solvent is dichloromethane.

36. The process of any one of claims 20-35, wherein R₁ is n-pentyl.

37. A process for producing a compound having the structure of formula (±)-(I): 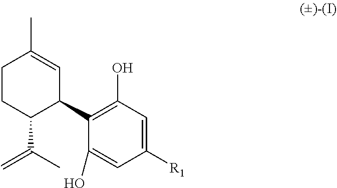
wherein
R₁ is alkyl,
the process comprising:
(i) reacting citral with a Lewis acid in a suitable solvent under conditions sufficient to produce a compound having the structure of formula (±)-(IIa):

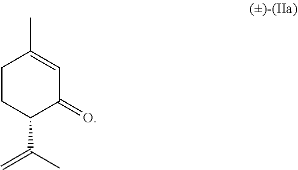

38. The process of claim 37, wherein the citral is a mixture of citral A and citral B.

39. The process of claim 37, wherein the citral is citral B.

40. The process of any one of claims 37-39, wherein the Lewis acid comprises an aluminum metal center.

41. The process of claim 40, wherein the Lewis acid is a dialkyl aluminum chloride.

42. The process of claim 41, wherein the Lewis acid is dimethyl aluminum chloride.

43. The process of claim 41, wherein the Lewis acid is diethyl aluminum chloride.

44. The process of any one of claims 37-43, wherein a solution of citral in the suitable solvent and a solution of the Lewis acid in the suitable solvent are slowly mixed together over 2 to 4 hours.

45. The process of claim 44, wherein the mixing occurs at a temperature of less than 0° C.

46. The process of claim 44 or 45, wherein the mixing occurs at a temperature of about-10° C.

47. The process of any one of claims 37-46, wherein the suitable solvent is dichloromethane.

48. The process of any one of claims 37-47, further comprising:
(ia) reacting the compound having the structure of formula (±)-(IIa) produced in step (i) with a reducing agent in a second suitable solvent under conditions sufficient to produce a compound having the structure of formula (+)-(II″):

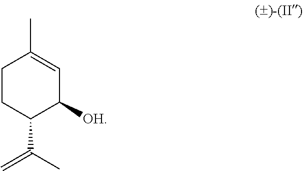

49. The process of claim 48, wherein the reducing agent comprises lithium metal.

50. The process of claim 49, wherein the reducing agent is lithium aluminum hydride.

51. The process of claim 49 or 50, wherein the second suitable solvent is tetrahydrofuran.

52. The process of any one of claims 48-51, wherein the reaction of step (ia) occurs at a temperature of less than 0° C.

53. The process of any one of claims 37-52, further comprising:
(ii) reacting the compound having the structure of formula (±)-(II) produced in step (i) with a compound having the structure of formula (III) in the presence of an acid in a second suitable solvent: 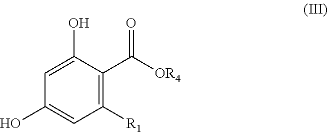
wherein R₄ is alkyl, under conditions sufficient to produce a compound having the structure of formula (±)-(IV):

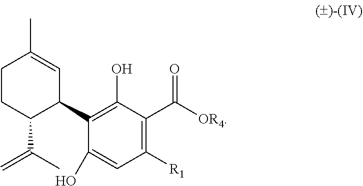

54. The process of claim 53, wherein the acid is an organic acid or a Lewis acid.

55. The process of claim 54, wherein the acid is camphorsulfonic acid.

56. The process of any one of claims 53-55, wherein the suitable second solvent is dichloromethane.

57. The process of any one of claims 53-56, further comprising:
(iii) subjecting the compound having the structure of formula (±)-(IV) produced in step (ii) to basic hydrolysis conditions sufficient to produce a compound having the structure of formula (±)-(V): 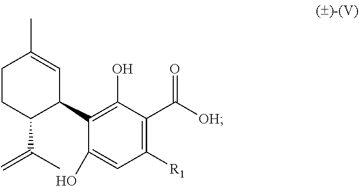
and
(iv) subjecting the compound having the structure of formula (±)-(V) produced in step (iii) to decarboxylation conditions sufficient to produce the compound having the structure of formula (±)-(I).

58. The process of any one of claims 53-57, wherein the compound having the structure of formula (III) is prepared by a process comprising:
(a) reacting a compound having the structure of formula (VIa): 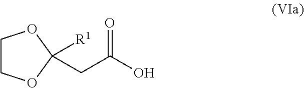
with an activating agent in a first suitable solvent under conditions sufficient to produce a the structure of formula (VIb): 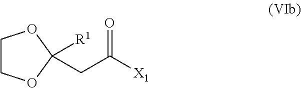
wherein
X₁ is a halide or activating ester moiety;
(b) reacting the compound having the structure of formula (VIb) with a diene having the structure of formula (VIc) in the presence of a Lewis acid in a second suitable solvent: 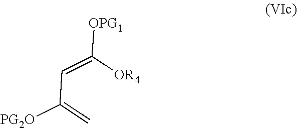
wherein
PG₁ and PG₂ are each independently a hydroxyl protecting group; and
R₄ is alkyl, under conditions sufficient to form the cyclized compound having the structure of formula (III).

59. The process of any one of claims 37-59, wherein R₁ is n-pentyl.

60. A process for producing a compound having the structure of formula (±)-(Ia): 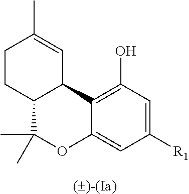
wherein
R₁ is alkyl,
the process comprising:
(i) reacting citral with a Lewis acid in a suitable solvent under conditions sufficient to produce a compound having the structure of formula (±)-(IIa):

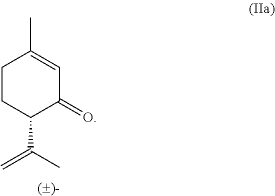

61. The process of claim 60, wherein the citral is a mixture of citral A and citral B.

62. The process of claim 61, wherein the citral is citral B.

63. The process of any one of claims 60-62, wherein the Lewis acid comprises an aluminum metal center.

64. The process of claim 63, wherein the Lewis acid is a dialkyl aluminum chloride.

65. The process of claim 64, wherein the Lewis acid is dimethyl aluminum chloride.

66. The process of claim 64, wherein the Lewis acid is diethyl aluminum chloride.

67. The process of any one of claims 60-66, wherein a solution of citral in the suitable solvent and a solution of the Lewis acid in the suitable solvent are slowly mixed together over 2 to 4 hours.

68. The process of claim 67, wherein the mixing occurs at a temperature of less than 0° C.

69. The process of claim 67 or 68, wherein the mixing occurs at a temperature of about-10° C.

70. The process of any one of claims 60-69, wherein the suitable solvent is dichloromethane.

71. The process of any one of claims 60-70, further comprising:
(ia) reacting the compound having the structure of formula (±)-(IIa) produced in step (i) with a reducing agent in a second suitable solvent under conditions sufficient to produce a compound having the structure of formula (+)-(II″):

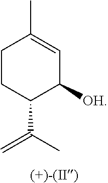

72. The process of claim 71, wherein the reducing agent comprises lithium metal.

73. The process of claim 72, wherein the reducing agent is lithium aluminum hydride.

74. The process of claim 72 or 73, wherein the second suitable solvent is tetrahydrofuran.

75. The process of any one of claims 71-74, wherein the reaction of step (ia) occurs at a temperature of less than 0° C.

76. The process of any one of claims 60-75, further comprising:
(ii) reacting the compound having the structure of formula (±)-(II) produced in step (i) with a compound having the structure of formula (IIIa) in the presence of an acid in a second suitable solvent: 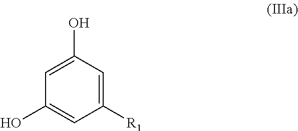
thereby producing the compound having the structure of formula (±)-(Ia).

77. The process of claim 76, wherein the acid is an organic acid or a Lewis acid.

78. The process of claim 77, wherein the acid is p-toluenesulfonic acid.

79. The process of any one of claims 76-78, wherein the suitable solvent is dichloromethane.

80. The process of any one of claims 60-79, wherein R₁ is n-pentyl.

81. A process for producing a compound having the structure of formula (±)-(IIa): 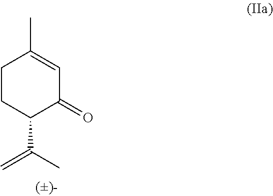
the process comprising reacting citral with a Lewis acid in a suitable solvent under conditions sufficient to produce the compound.

82. The process of claim 81, wherein the citral is a mixture of citral A and citral B.

83. The process of claim 81, wherein the citral is citral B.

84. The process of any one of claims 81-83, wherein the Lewis acid comprises an aluminum metal center.

85. The process of claim 84, wherein the Lewis acid is a dialkyl aluminum chloride.

86. The process of claim 85, wherein the Lewis acid is dimethyl aluminum chloride.

87. The process of claim 85, wherein the Lewis acid is diethyl aluminum chloride.

88. The process of any one of claims 81-87, wherein a solution of citral in the suitable solvent and a solution of the Lewis acid in the suitable solvent are slowly mixed together over 2 to 4 hours.

89. The process of claim 88, wherein the mixing occurs at a temperature of less than 0° C.

90. The process of claim 88 or 89, wherein the mixing occurs at a temperature of about-10° C.

91. The process of any one of claims 81-90, wherein the suitable solvent is dichloromethane.

92. A process for producing a compound having the structure of formula (±)-(II″): 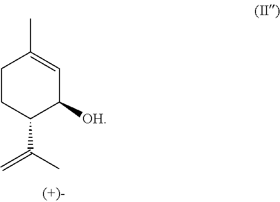
the process comprising preparing the compound having the structure of formula (±)-(IIa) according to the process of any one of claims 81-90; and reacting the compound having the structure of formula (±)-(IIa) with a reducing agent in a second suitable solvent under conditions sufficient to produce the compound having the structure of formula (±)-(II″).

93. A process for producing a compound having the structure of formula (+)-(II′) or (+)-(II″): 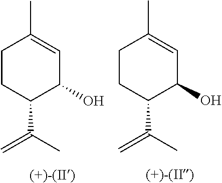
the process comprising:
(i) reacting (R)-limonene with an oxidizing agent in a first suitable solvent under conditions sufficient to produce a compound having the structure of formula (+)-(IIa): 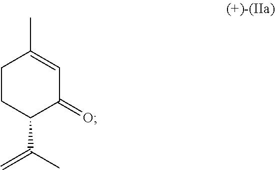
and
(ii) reacting the compound having the structure of formula (+)-(IIa) produced in step (i) with a reducing agent in a second suitable solvent under conditions sufficient to produce a compound having the structure of formula (+)-(II′) as a single enantiomer or (+)-(II″) as a single enantiomer.

94. The process of claim 93, wherein the oxidizing agent comprises a chromium metal complex or salt.

95. The process of claim 94, wherein the oxidizing agent is chromium (VI) oxide.

96. The process of any one of claims 93-95, wherein the first suitable solvent is chloroform.

97. The process of any one of claims 93-96, wherein the reducing agent comprises sodium metal.

98. The process of claim 97, wherein the reducing agent is sodium borohydride.

99. The process of claim 97 or 98, wherein the second suitable solvent is methanol.

100. The process of any one of claims 97-99, wherein the reaction of step (ii) occurs in the presence of cerium (III) chloride or samarium (III) iodide.

101. The process of any one of claims 93-96, wherein the reducing agent comprises lithium metal.

102. The process of claim 101, wherein the reducing agent is lithium aluminum hydride.

103. The process of claim 101 or 102, wherein the second suitable solvent is tetrahydrofuran.

104. The process of any one of claims 101-103, wherein the reaction of step (ii) occurs at a temperature of less than 0° C.

105. The process of any one of claims 57, 76, and 93-104, wherein only (+)-(II″) is produced in step (ii).

106. A process for producing a compound having the structure of formula (III) 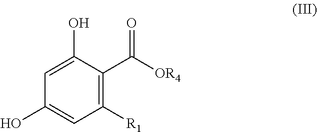
wherein
R₁ is alkyl; and
R₄ is alkyl,
the process comprising:
(i) reacting the compound having the structure of formula (VII): 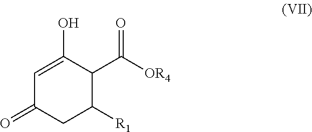
with Br₂ in a first suitable solvent under conditions sufficient to produce the compound having the structure of formula (VIIa):

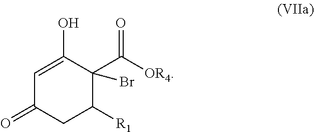

107. The process of claim 106, wherein the first suitable solvent is dimethylformamide.

108. The process of claim 106 or 107, wherein the reaction occurs at a temperature of −10 to 10° C.

109. The process of any one of claims 106-108, wherein the reaction occurs at a temperature of −10 to 0° C.

110. The process of any one of claims 106-109, wherein about 1 molar equivalent of Br₂ is employed in the reaction.

111. The process of any one of claims 106-110, further comprising:
(ii) refluxing the compound having the structure of formula (VIIa) produced in step (i) in a second suitable solvent under conditions sufficient to produce a compound having the structure of formula (III).

112. The process of claim 111, wherein the second suitable solvent is toluene.

113. The process of any one of claims 106-112, wherein the compound having the structure of formula (VII) is prepared by a process comprising reacting dimethyl malonate with trans-3-nonen-2-one in the presence of a base in a third suitable solvent under conditions sufficient to produce the compound having the structure of formula (VII).

114. The process of claim 113, wherein the base is sodium methoxide.

115. The process of claim 113 or 114, wherein the third suitable solvent is methanol.

116. The process of any one of claims 113-115, wherein the reaction occurs at reflux.

117. A process for producing a compound having the structure of formula (III) 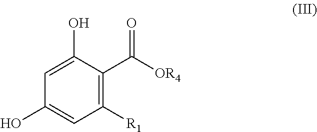
wherein
R₁ is alkyl; and
R₄ is alkyl,
the process comprising:
(b) reacting the compound having the structure of formula (VIb) 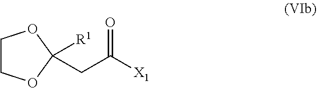
wherein
X₁ is a halide or activating ester moiety,
with a diene having the structure of formula (VIc) in the presence of a Lewis acid in a suitable solvent: 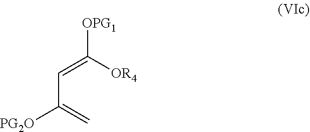
wherein
PG₁ and PG₂ are each independently a hydroxyl protecting group; and
R₄ is alkyl, under conditions sufficient to form the cyclized compound having the structure of formula (III).

118. The process of claim 117, further comprising:
(a) reacting a compound having the structure of formula (VIa): 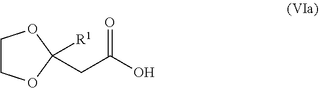
with an activating agent in a first suitable solvent under conditions sufficient to produce a compound having the structure of formula (VIb): 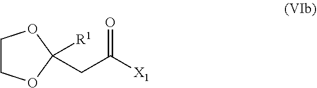
wherein
X₁ is a halide or activating ester moiety.

119. The process of claim 118, wherein in step (a) the activating agent is oxalyl chloride.

120. The process of claim 119, wherein the first suitable solvent is benzene.

121. The process of any one of claims 117-120, wherein in step (b) the Lewis acid is titanium chloride.

122. The process of claim 121, wherein the suitable solvent is dichloromethane.

123. The process of any one of claims 106-116, wherein R₄ is methyl.

124. The process of any one of claims 117-123, wherein R₁ is n-pentyl.

125. A process for producing a compound having the structure of formula (VIII) 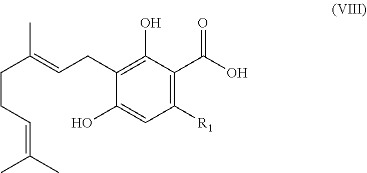
wherein
R₁ is alkyl; and
R₄ is alkyl,
the process comprising:
(i) reacting the compound having the structure of formula (III): 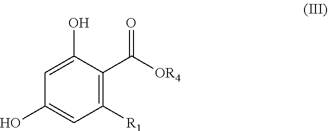
with geraniol in the presence of an acid in a first suitable solvent under conditions sufficient to produce the compound having the structure of formula (VIIIa):

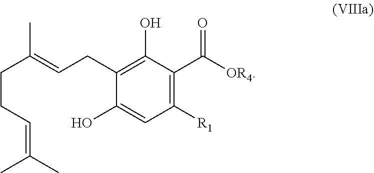

126. The process of claim 125, wherein the acid is an organic acid or a Lewis acid.

127. The process of claim 126, wherein the acid is camphorsulfonic acid or p-toluenesulfonic acid.

128. The process of any one of claims 125-127, wherein the first suitable solvent is dichloromethane.

129. The process of any one of claims 125-128, wherein the reaction occurs at a temperature of 0 to 25° C.

130. The process of any one of claims 125-129, further comprising:
(ii) subjecting the compound having the structure of formula (VIIIa) prepared in step (i) to decarboxylation conditions sufficient to produce the compound having the structure of formula (VIII).

131. The process of any one of claims 125-130, wherein R₄ is methyl.

132. The process of any one of claims 125-131, wherein R₁ is n-pentyl.

133. The process of any one of claims 125-132, wherein the compound of formula (III) is produced according to the process of any one of claims 106-116.

134. The process of any one of claims 125-132, wherein the compound of formula (III) is produced according to the process of any one of claims 117-122.

135. The process of any one of claims 37-59, wherein the compound produced has the structure:

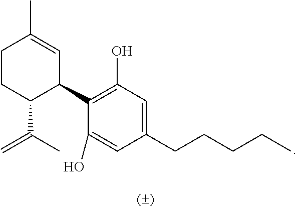 [SMILES: C=C(C)[C@@H]1CCC(C)=C[C@H]1c1c(O)cc(CCCCC)cc1O]

136. The process of any one of claims 60-80, wherein the compound produced has the structure:

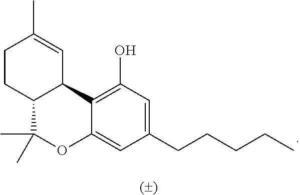 [SMILES: CCCCCc1cc(O)c2c(c1)OC(C)(C)[C@@H]1CCC(C)=C[C@@H]21]

137. The process of any one of claims 1-19, wherein the compound produced has the structure:

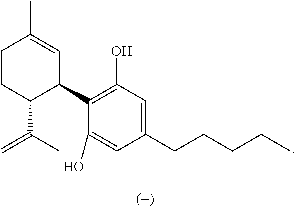 [SMILES: C=C(C)[C@@H]1CCC(C)=C[C@H]1c1c(O)cc(CCCCC)cc1O]

138. The process of any one of claims 20-36, wherein the compound produced has the structure:

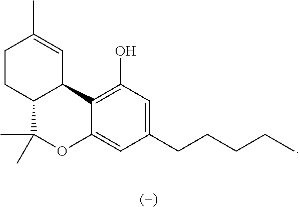 [SMILES: CCCCCc1cc(O)c2c(c1)OC(C)(C)[C@@H]1CCC(C)=C[C@@H]21]

139. The process of any one of claims 125-134, wherein the compound produced has the structure:

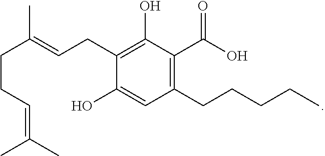 [SMILES: CCCCCc1cc(O)c(C/C=C(\C)CCC=C(C)C)c(O)c1C(=O)O]

140. A process for preparing (±)-cannabidiol (CBD), wherein the process comprises a preparation of (±)-isopiperitenone according to any one of claims 81-91.

141. A process for preparing (±)-delta-9-tetrahydrocannabinol (Δ⁹-THC), wherein the process comprises a preparation of (±)-isopiperitenone according to any one of claims 81-91.

142. A process for preparing (−)-cannabidiol (CBD), wherein the process comprises a preparation of (+)-trans-isopiperitenol according to any one of claims 93-105.

143. A process for preparing (−)-delta-9-tetrahydrocannabinol (Δ⁹-THC), wherein the process comprises a preparation of (+)-trans-isopiperitenol according to any one of claims 93-105.

144. A pharmaceutical composition comprising (±)-cannabidiol (CBD) and a pharmaceutically acceptable carrier, wherein the (±)-cannabidiol (CBD) is produced according to the process of any one of claim 1-19 or 37-59.

145. A pharmaceutical composition comprising (±)-delta-9-tetrahydrocannabinol (49-THC) and a pharmaceutically acceptable carrier, wherein the (±)-delta-9-tetrahydrocannabinol (Δ⁹-THC) is produced according to the process of any one of claim 20-36 or 60-80.

146. A compound having the structure: 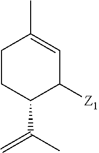
wherein Z₁ is OH, OMs, OTs, or halo.

147. The compound of claim 146 having the structure: 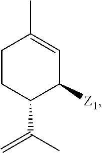
wherein the compound has the absolute configuration as shown.

148. The compound of claim 146 having the structure: 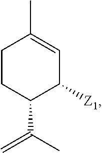
wherein the compound has the absolute configuration as shown,
